(19) United States
(12) Patent Application Publication
(10) Pub. No.: US 2009/0254124 A1
(43) Pub. Date: Oct. 8, 2009

(54) METHOD AND APPARATUS FOR SECURING AN OBJECT TO BONE AND/OR FOR STABILIZING BONE

(76) Inventors: Barry T. Bickley, North Andover, MA (US); Aldo M. Zovich, East Hampton, CT (US); Richard E. Zovich, Kensington, CT (US)

Correspondence Address: Mark J. Pandiscio;Pandiscio & Pandiscio, P.C., 470 Totten Pond Road, Waltham, MA, 02451, US

(21) Appl. No.: 12/268,140

(22) Filed: Nov. 10, 2008

Related U.S. Application Data
(63) Continuation-in-part of application No. 10/554,379, filed on Oct. 25, 2005.
(60) Provisional application No. 60/468,829, filed on May 8, 2003.

(51) Int. Cl. A61B 17/70; A61B 17/86; A61B 17/88

(52) U.S. Cl. 606/264; 606/302; 606/279

(57) ABSTRACT

A surgical system comprising:
an object to be secured to bone, the object comprising an opening extending therethrough; and
a sleeve/screw construction for securing the object to bone, the sleeve/screw construction comprising:
   a sleeve adapted for positioning through the opening in the object and into the bone, the sleeve comprising:
      a shank having a distal end and a proximal end and an opening extending from the distal end to the proximal end, wherein the opening narrows toward the distal end of the shank, and further wherein the distal end of the shank is slit so as to form a plurality of radially-expandable segments; and
      an enlarged head formed at the proximal end of the shank, the enlarged head being formed so as to be radially-expandable;
      the sleeve being sized so that when the sleeve is positioned through the opening in the object and into the bone, at least a portion of the radially-expandable segments extend into the bone beyond the cortical bone/cancellous bone interface; and
   a screw adapted for positioning through the opening in the sleeve, the screw being sized so as to (i) radially expand the distal end of the sleeve so that the sleeve is secured to the bone, and (ii) radially expand the enlarged head of

the sleeve so that the sleeve is secured to the object, whereby to secure the object to the bone.

### REFERENCE TO PENDING PRIOR PATENT APPLICATIONS

This patent application:

(1) is a continuation-in-part of pending prior U.S. patent application Ser. No. 10/554,379, filed Oct. 25, 2005 by Barry T. Bickley et al. for FIXATION AUGMENTATION DEVICE AND RELATED TECHNIQUES (Attorney's Docket No. BICK-002BUS), which in turn claims benefit of:
(a) International (PCT) Patent Application No. PCT/US04/14640, filed May 10, 2004 for FIXATION AUGMENTATION DEVICE AND RELATED TECHNIQUES (Attorney's Docket No. BICK-002 BWO), which itself claims benefit of U.S. Provisional Patent Application Ser. No. 60/468,829, filed May 8, 2003 for FIXATION AUGMENTATION DEVICE (Attorney's Docket No. BICK-005 PUSP); and
(b) U.S. patent application Ser. No. 10/246,304, filed Sep. 18, 2002 for FIXATION AUGMENTATION DEVICE AND RELATED TECHNIQUES (Attorney's Docket No. BICK-002 AUS);

(2) is a continuation-in-part of pending prior U.S. patent application Ser. No. 12/148,845, filed Apr. 23, 2008 by Barry T. Bickley et al. for METHOD AND APPARATUS FOR SECURING AN OBJECT TO BONE (Attorney's Docket No. SIM-1);

(3) is a continuation-in-part of pending prior U.S. patent application Ser. No. 12/156,495, filed Jun. 2, 2008 by Barry T. Bickley et al. for METHOD AND APPARATUS FOR SECURING AN OBJECT TO BONE AND/OR FOR STABILIZING BONE (Attorney's Docket No. SIM-2); and

(4) claims benefit of pending prior U.S. Provisional Patent Application Ser. No. 61/002,702, filed Nov. 9, 2007 by Barry T. Bickley et al. for METHOD AND APPARATUS FOR STABILIZING BONE (Attorney's Docket No. SIM-3 PROV).

The seven above-identified patent applications are hereby incorporated herein by reference.

### FIELD OF THE INVENTION

This invention relates to surgical methods and apparatus in general, and more particularly to surgical methods and apparatus for securing an object to bone and/or for stabilizing bone.

### BACKGROUND OF THE INVENTION

In many situations an object may need to be secured to bone. By way of example but not limitation, where a bone is fractured, it may be desirable to stabilize the bone with a bone plate which extends across the fracture line. By way of further example but not limitation, where two separate bones need to be secured together (e.g., in the case of a spinal fusion), it may be desirable to secure the two bones to one another with a bone plate which extends from one bone to the other. By way of still further example but not limitation, where soft tissue needs to be attached (or re-attached) to bone (e.g., in the case of a ligament repair or reconstruction), it may be desirable to capture the soft tissue to the bone using a fixation plate.

In all of the foregoing situations, as well as many others which are well known to those skilled in the art, a plate or other object needs to be secured to bone. Such attachment is most commonly effected by using a surgical screw which passes through a hole in the plate (or other object) and into the bone.

When using a surgical screw to secure a plate to bone, the plate is first aligned with the bone. Then a hole is drilled into the bone, by passing a drill through a pre-existing hole in the plate and into the bone. Next, the bone hole may be tapped. Then the surgical screw is passed through the hole in the plate and into the hole in the bone, whereby to secure the plate to the bone.

One problem which can arise during the foregoing procedure is that the hole in the bone may become stripped as the screw is inserted into the bone. When this occurs, the screw can no longer obtain adequate purchase in the bone, thereby undermining plate fixation. A screw having inadequate purchase is sometimes referred to as a “spinner”. Spinners can occur for many reasons, including (i) inadequate bone quality, (ii) over-tightening of the screw, (iii) an error when drilling the hole in the bone, (iv) an error when tapping the hole in the bone, etc. As noted above, spinners generally result in inadequate fixation.

### SUMMARY OF THE INVENTION

The present invention is intended to address the foregoing deficiencies of the prior art, by providing a new and improved method and apparatus for securing an object to bone and/or for stabilizing bone.

More particularly, the present invention provides a new and improved fixation system for securing an object to bone and/or for stabilizing bone.

In one preferred form of the present invention, the new fixation system comprises a plate which is to be secured to bone, and a sleeve and a screw for securing the plate to the bone. The plate comprises an opening which extends through the plate. The plate is placed against the bone and then a drill is used to form a hole in the bone beneath the opening. A sleeve is passed through the opening and into the hole in the bone. The sleeve and plate are formed so that the sleeve (and the recipient bone hole) can be disposed at any one of a variety of angles relative to the plate. A screw is then passed through the sleeve, radially expanding the sleeve so that the sleeve is simultaneously secured to both the bone and the plate.

In another preferred form of the present invention, the new fixation system is intended to stabilize bone in general, and vertebral bodies in particular.

In a preferred form of the present invention, there is provided a novel anterior cervical plate (ACP) system which comprises a novel ACP which is to be attached to two adjacent cervical bodies, and attachment apparatus for attaching the ACP to the two cervical bodies. Preferably, the attachment apparatus comprise a screw and, in one preferred form of the invention, the attachment apparatus comprise a sleeve and screw combination, where the sleeve acts as an interface between (i) the bone and the screw, and (ii) the ACP and the screw, with the sleeve enhancing fixation. Among other things, the ACP is specifically configured to provide the option of adding future level extensions.

In one form of the invention, there is provided a surgical system comprising:
an object to be secured to bone, the object comprising an opening extending therethrough; and
a sleeve/screw construction for securing the object to bone, the sleeve/screw construction comprising:
   a sleeve adapted for positioning through the opening in the object and into the bone, the sleeve comprising:
      a shank having a distal end and a proximal end and an opening extending from the distal end to the proximal end, wherein the opening narrows toward the distal end of the shank, and further wherein the distal end of the shank is slit so as to form a plurality of radially-expandable segments; and
      an enlarged head formed at the proximal end of the shank, the enlarged head being formed so as to be radially-expandable;
      the sleeve being sized so that when the sleeve is positioned through the opening in the object and into the bone, at least a portion of the radially-expandable segments extend into the bone beyond the cortical bone/cancellous bone interface; and
   a screw adapted for positioning through the opening in the sleeve, the screw being sized so as to (i) radially expand the distal end of the sleeve so that the sleeve is secured to the bone, and (ii) radially expand the enlarged head of the sleeve so that the sleeve is secured to the object, whereby to secure the object to the bone.

In another form of the invention, there is provided a method for securing an object to bone, the method comprising the steps of:
providing an object having an opening extending therethrough, and providing a sleeve/screw construction for securing the object to bone, the sleeve/screw construction comprising:
   a sleeve adapted for positioning through the opening in the object and into the bone, the sleeve comprising:
      a shank having a distal end and a proximal end and an opening extending from the distal end to the proximal end, wherein the opening narrows toward the distal end of the shank, and further wherein the distal end of the shank is slit so as to form a plurality of radially-expandable segments;
      an enlarged head formed at the proximal end of the shank, the enlarged head being formed so as to be radially-expandable;
      the sleeve being sized so that when the sleeve is positioned through the opening in the object and into the bone, at least a portion of the radially-expandable segments extend into the bone beyond the cortical bone/cancellous bone interface; and
   a screw adapted for positioning through the opening in the sleeve, the screw being sized so as to (i) radially expand the distal end of the sleeve so that the sleeve is secured to the bone, and (ii) radially expand the enlarged head of the sleeve so that the sleeve is secured to the object, whereby to secure the object to the bone;
positioning the object against the bone;
placing the sleeve through the opening in the object and into the bone; and
positioning the screw in the sleeve so as to secure the sleeve to the bone and to the object, whereby to secure the object to the bone.

### BRIEF DESCRIPTION OF THE DRAWINGS

These and other objects and features of the present invention will be more fully disclosed or rendered obvious by the following detailed description of the preferred embodiments of the invention, which is to be read in conjunction with the attached drawings wherein like numbers refer to like parts, and further wherein:

FIG. 1 is a schematic view showing one preferred form of the novel fixation system of the present invention;

FIGS. 2 and 3 are schematic views showing one preferred form of a plate;

FIG. 4 is a schematic view showing an alternative form of the novel fixation system;

FIGS. 5-10 are schematic views showing one preferred form of a sleeve;

FIGS. 11-14 are schematic views showing one preferred form of a screw;

FIGS. 15-20 are schematic views showing a plate being secured to a bone using a plurality of sleeve/screw constructions;

FIGS. 21-25 are schematic views showing another preferred form of a plate;

FIGS. 26-28 are schematic views showing another preferred form of a sleeve;

FIG. 29 is a schematic view showing another preferred form of a screw;

FIG. 30 is a schematic view showing a rod for use with the sleeve/screw construction of the present invention;

FIG. 31 is a schematic view showing another form of rod for use with the sleeve/screw construction of the present invention;

FIG. 32 is a schematic view of a plate for capturing a rod against bone;

FIG. 33 is a schematic view of a “tulip” mount which may be secured to a bone using the sleeve/screw construction of the present invention;

FIGS. 34 and 35 show the sleeve being mated with the tulip mount, and the screw being mated with the sleeve, respectively;

FIG. 36 is a schematic view showing a hybrid tulip mount/sleeve construction;

FIG. 37 is a schematic view showing a screw being mated with the hybrid tulip mount/sleeve construction shown in FIG. 36;

FIG. 38 is a schematic top perspective view illustrating (i) a primary anterior cervical plate (ACP) formed in accordance with the present invention, and (ii) a supplemental ACP formed in accordance with the present invention;

FIG. 39 is an enlarged schematic top perspective view illustrating the primary ACP shown in FIG. 38;

FIG. 40 is an enlarged schematic top perspective view illustrating the supplemental ACP shown in FIG. 38;

FIG. 41 is a schematic bottom perspective view illustrating the primary ACP and the supplemental ACP shown in FIG. 38;

FIG. 42 is a schematic end perspective view illustrating the primary ACP and the supplemental ACP shown in FIG. 38;

FIGS. 43-46 are schematic views of the preferred form of attachment apparatus used to secure the primary ACP and the supplemental ACP to bone;

FIG. 47 is a schematic side view showing how a supplemental ACP 700 fits over the primary ACP 600;

FIGS. 48 and 49 are schematic top views showing how a primary ACP 600 and a supplemental ACP 700 may be oriented “off-axis” to one another;

FIGS. 50 and 51 are schematic side views showing how attachment apparatus 635 may pivot relative to primary ACP 600;

FIG. 52 is a schematic side view showing how attachment apparatus 635 may translate longitudinally relative to primary ACP 600;

FIG. 53 is a schematic top perspective view illustrating a primary ACP 600 with a protective collar attached;

FIG. 54 is a schematic top perspective view illustrating a supplemental ACP 700 with a protective collar attached;

FIGS. 55-58 are schematic views showing an alternative form of the novel fixation system;

FIG. 59 is a schematic view showing one preferred form of a plate;

FIGS. 60-64 are schematic views showing one preferred form of a sleeve;

FIGS. 65-67 are schematic views showing one preferred form of a screw; and

FIGS. 68-74 are schematic views showing an alternative form of the novel fixation system.

### DETAILED DESCRIPTION OF THE PREFERRED EMBODIMENTS

### A Novel Fixation System Comprising a Plate, a Sleeve and a Screw for Securing an Object to Bone

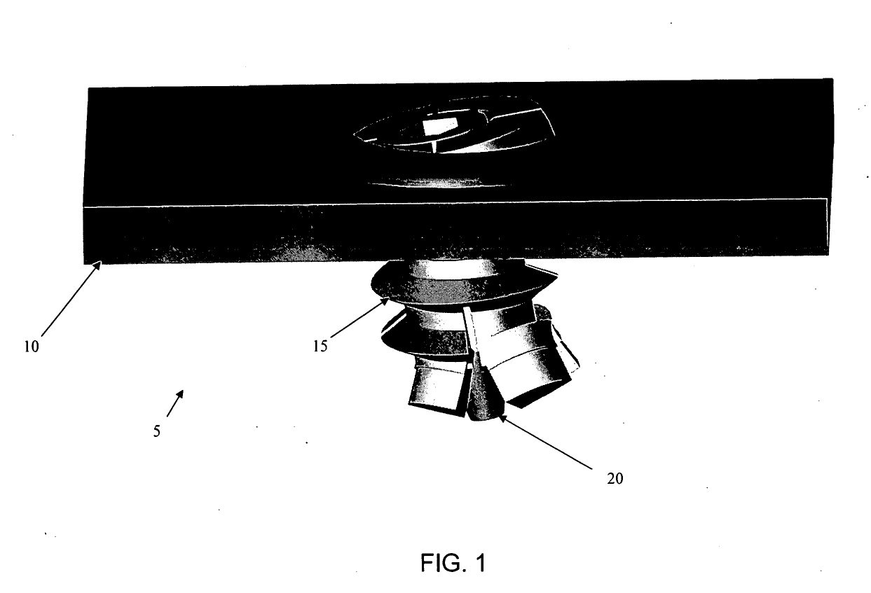

Looking first at FIG. 1, there is shown a novel fixation system 5 which generally comprises a plate 10 which is to be secured to bone, a sleeve 15 and a screw 20 for securing plate 10 to the bone.

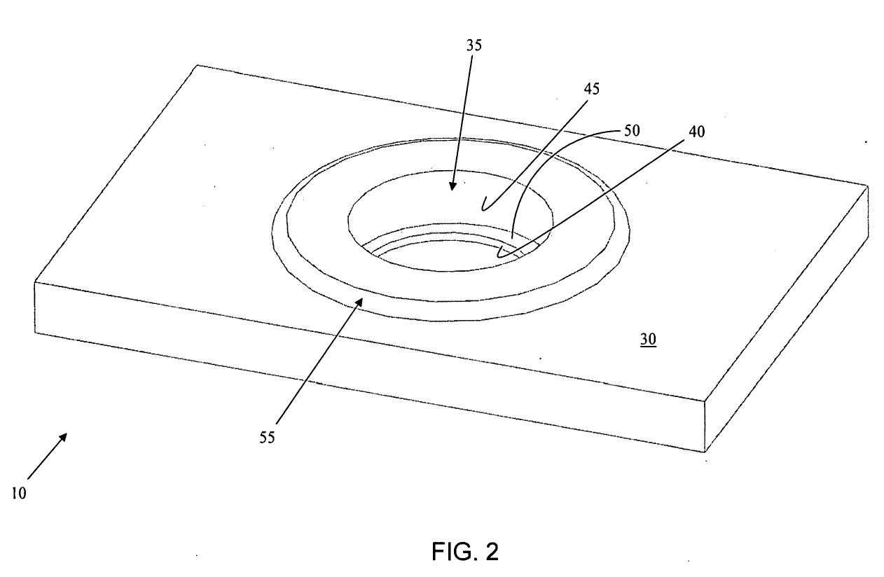

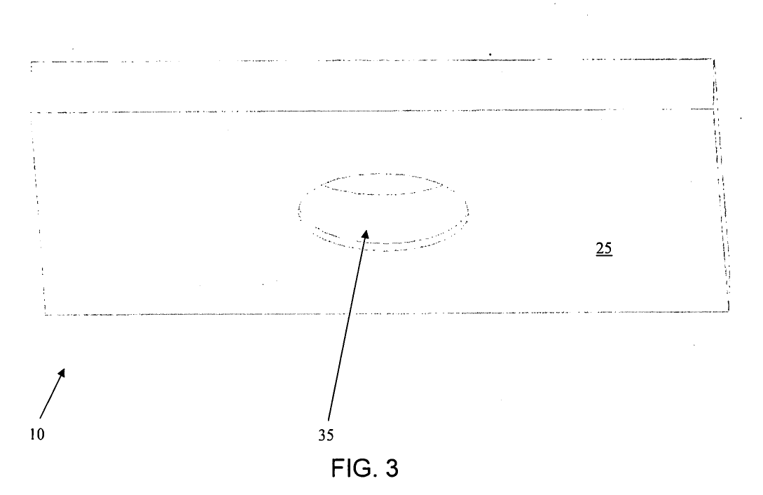

Plate 10 is shown in detail in FIGS. 2 and 3. Plate 10 generally comprises a distal surface 25 (FIG. 3) for positioning against bone, a proximal surface 30 (FIG. 2), and at least one opening 35 formed in the plate. Opening 35 is preferably in the form of a bore-counterbore configuration, i.e., a bore 40 opens on distal surface 25, a counterbore 45 opens on proximal surface 30, and an annular flange 50 is formed at the intersection of bore 40 and counterbore 45. As will hereinafter be discussed in further detail, bore 40 is sized to receive the shank of sleeve 15, and counterbore 45 is sized to receive the head of sleeve 15, with annular flange 50 serving to support the head of sleeve 15 and prevent the head of the sleeve from passing through the plate.

Opening 35 is preferably dimensioned, and one or more of the plate surfaces defining opening 35 are preferably appropriately radiused, and counterpart portions of sleeve 15 are preferably appropriately radiused, in order to permit sleeve 15 to extend through plate 10 at a range of different angles as will hereinafter be discussed in further detail. See, for example, FIG. 1, where sleeve 15 is shown extending through plate 10 at an acute angle.

A raised rim 55 is preferably formed on proximal surface 30 adjacent to opening 35. Raised rim 55 helps to present a smooth interface between the elements of the system and the surrounding tissue, particularly when sleeve 15 and screw 20 are placed at an acute angle relative to the plane of plate 10 (i.e., at an angle significantly off the perpendicular, such as is shown in FIG. 1). In addition, raised rim 55 also provides an enlarged contact surface for the head of sleeve 15, particularly when sleeve 15 and screw 20 are placed at an acute angle relative to the plane of plate 10 (i.e., an angle significantly off the perpendicular). See, for example, FIG. 1.

Depending on the intended use of plate 10, more than one opening 35 may be provided. By way of example but not limitation, where plate 10 is intended to be used as a fracture fixation plate or as a spinal fusion plate, at least one (and preferably two or more) openings 35 are formed in plate 10 on either side of the bone separation line (e.g., the fracture line, the vertebral body abutment lines, etc.), such that plate 10 can be secured to bone on each side of the bone separation line. By way of further example but not limitation, where plate 10 is intended to be used to secure soft tissue to bone, plate 10 might include only one opening 35.

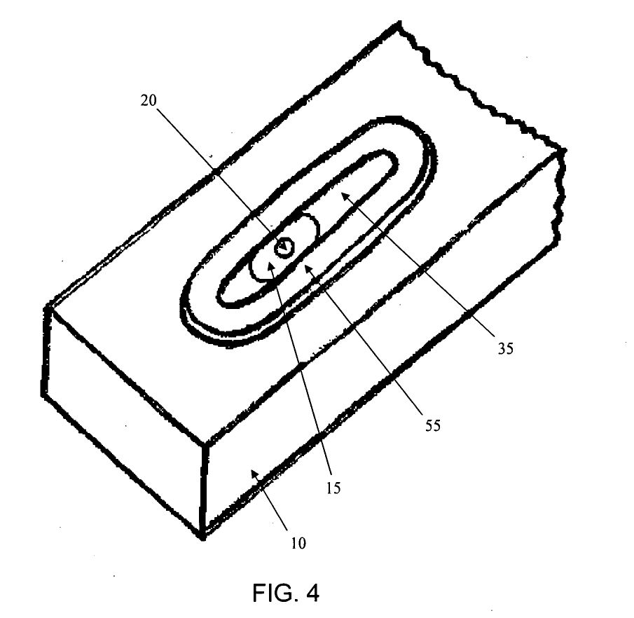

If desired, opening 35 in plate 10 and head 65 of sleeve 15 may be formed with non-circular (e.g., oval) shapes (as seen in top view) so as to provide an anti-rotation contact between the sleeve and the plate. Furthermore, if desired, opening 35 in plate 10 can have a slot-like configuration (as seen in top view), so as to allow a degree of longitudinal freedom when determining where to place sleeve 15 through opening 35 in plate 10. See FIG. 4.

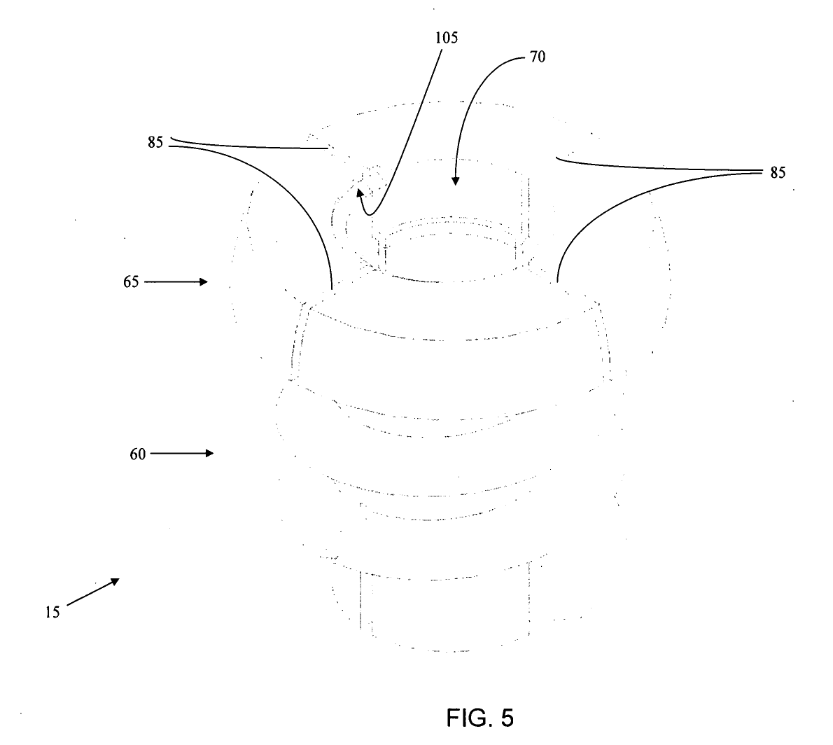

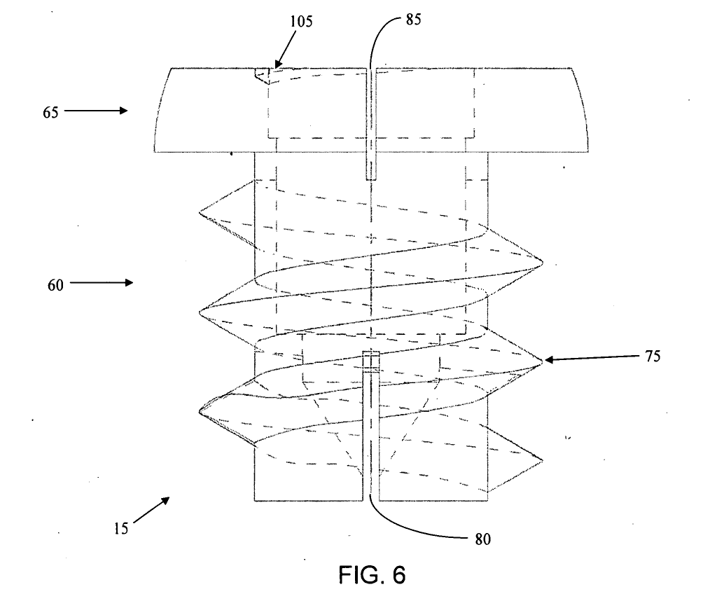

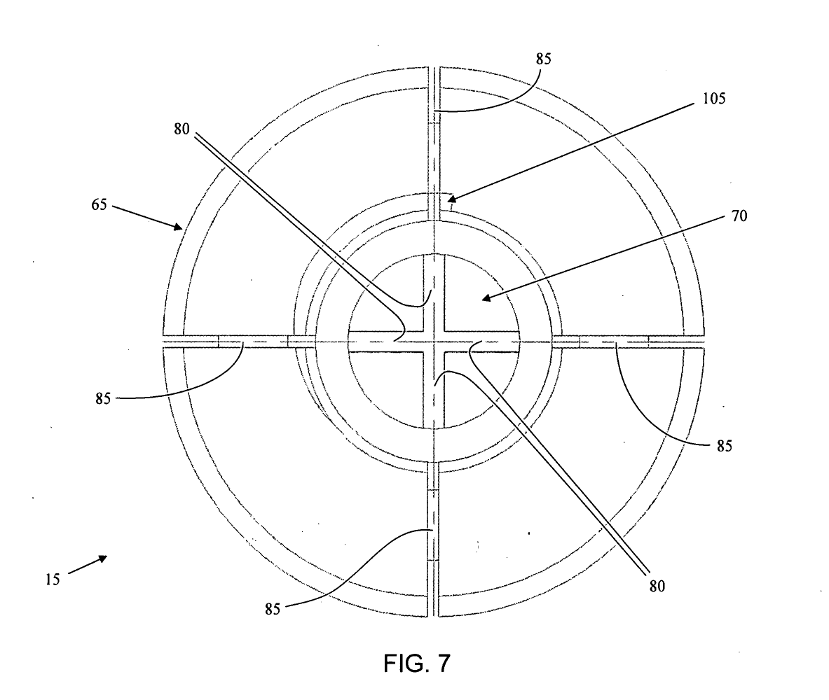

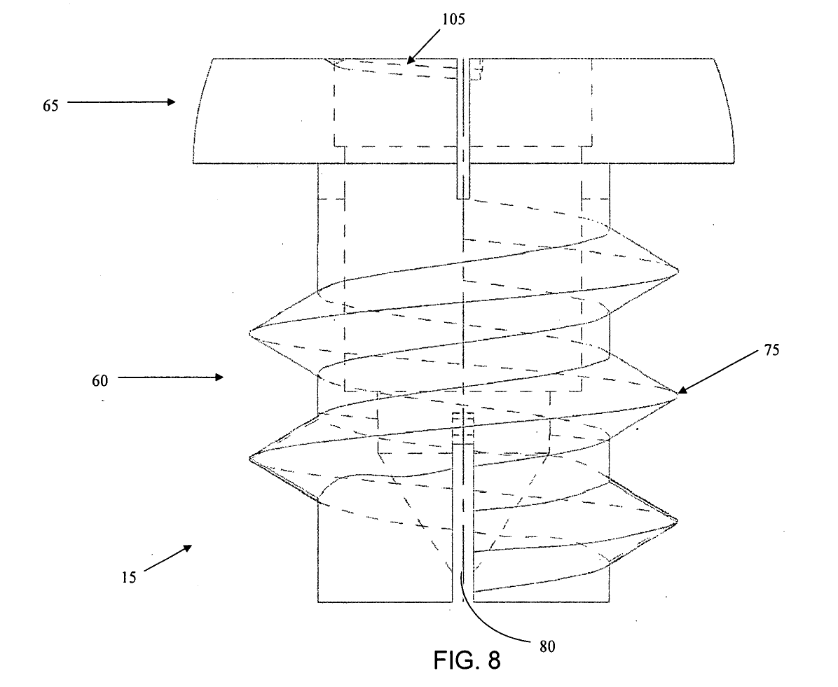

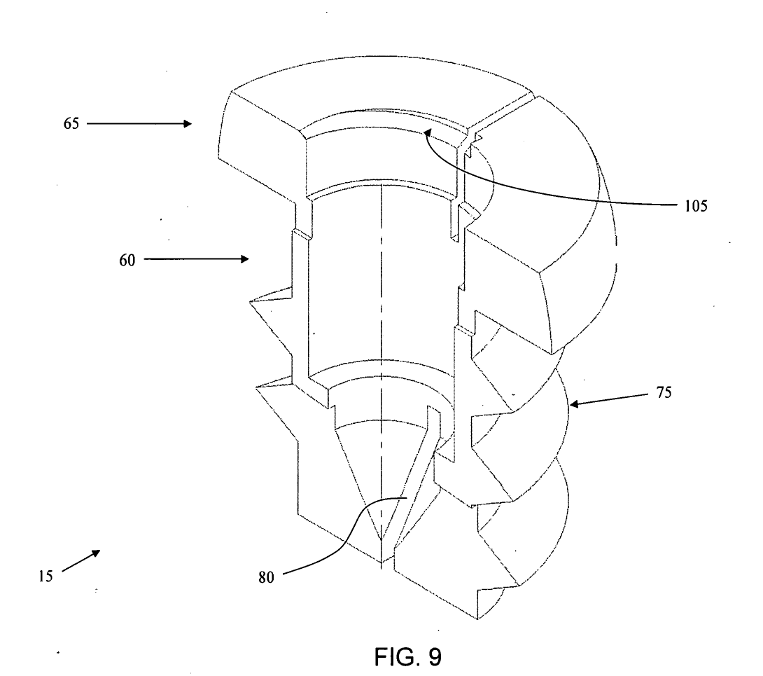

Sleeve 15 is shown in detail in FIGS. 5-10. Sleeve 15 generally comprises a shank 60, a head 65 and an opening 70 extending along the length of sleeve 15.

Shank 60 comprises a screw thread 75 on its outer surface. Screw thread 75 is preferably configured to facilitate the gripping entry of sleeve 15 into bone when the sleeve is turned into bone. Such screw threads may be self-drilling, in which case it may not be necessary to pre-drill a hole in the bone. Furthermore, the threads may be self-tapping, or they may not be self-tapping, in which case it may be necessary to tap a bone hole before inserting the sleeve into that bone hole. Sleeve 15 may be formed with threads having a reverse face so as to aid in backing the sleeve out of the bone, in the event that the same should be desired (e.g., in the case of a revision).

A plurality of slits 80 extend through the side wall of shank 60 at the distal end of shank 60. Slits 80 permit shank 60 to expand radially when screw 20 is disposed in opening 70, as will hereinafter be discussed in further detail.

Head 65 includes a plurality of longitudinally-extending slots 85. Slots 85 permit sleeve 15 to be held against rotation as screw 20 is turned into the sleeve, as will hereinafter be discussed in further detail. Slots 85 also permit head 65 to expand when screw 20 is turned into the sleeve, whereby to facilitate head 65 gripping adjacent portions of plate 10, as will hereinafter be discussed in further detail. Additionally, the head of sleeve 15 can be formed with a beveled edge so that it stands less proud when the sleeve is inserted into plate 10 at an angle which is relatively far off the perpendicular.

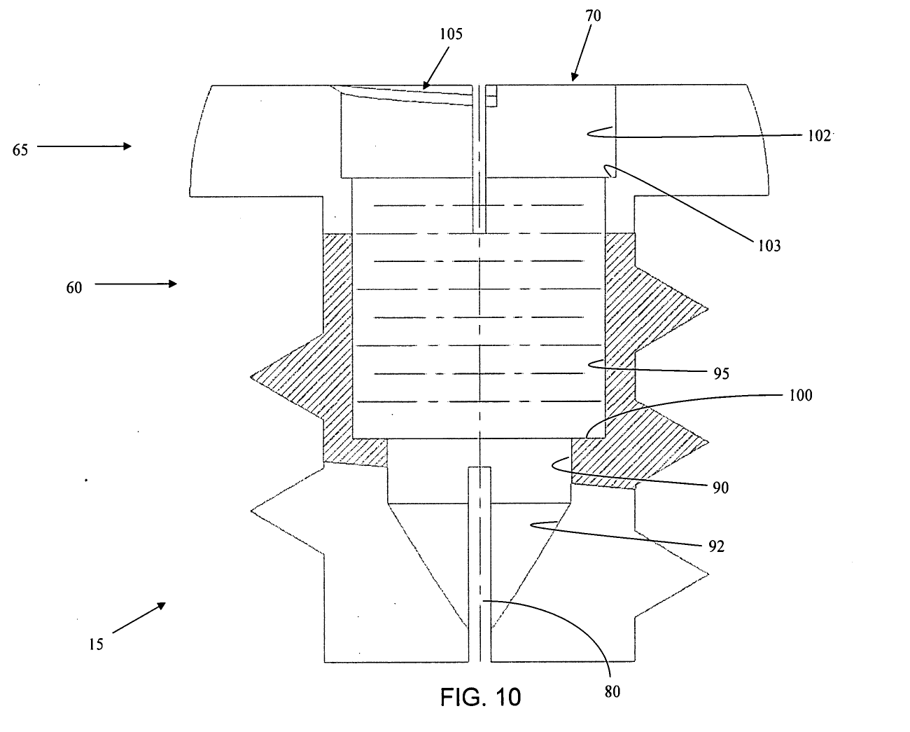

Opening 70 comprises a bore-counterbore-counterbore configuration. More particularly, and looking now at FIG. 10, a bore 90, terminating in a tapered portion 92, communicates with distal slits 80. A counterbore 95 communicates with bore 90. An annular flange 100 is formed at the intersection of bore 90 and counterbore 95. Another counterbore 102 communicates with counterbore 95 and opens on the proximal end of sleeve 15. An annular shoulder 103 is formed at the intersection of counterbore 95 and counterbore 102. As will hereinafter be discussed, counterbore 95 is sized to receive the shank of screw 20, and counterbore 102 is sized to receive the head of screw 20, with annular shoulder 103 serving to support the head of screw 20. However, sleeve 15 and screw 20 are sized so that when screw 20 is received in opening 70 of sleeve 15, engagement of the shank of screw 20 with tapered portion 92 of sleeve 15 will radially expand the distal end of sleeve 15 so as to grip the bone. Furthermore, sleeve 15 and screw 20 are also sized so that when the head of screw 20 is seated in counterbore 102, screw 20 will radially expand head 65 of sleeve 15 so as to grip plate 10.

It should be appreciated that (i) the size and shape of the head of screw 20, (ii) the size and shape of counterbore 102, and (iii) the size and shape of slots 85 in the head of sleeve 15, can all be combined so as to “tune” the degree of expansion of head 65 of sleeve 15, whereby to regulate the force with which the sleeve is secured to plate 10.

It should also be appreciated that (i) the size and shape of the shank of screw 20, (ii) the size and shape of counterbore 92, and (iii) the size and shape of slits 80, can all be combined so as to “tune” the degree of expansion of the distal end of sleeve 15, whereby to regulate the force with which the sleeve is secured to bone.

In addition to the foregoing, and as will hereinafter be discussed in further detail, sleeve 15 is preferably sized so that, when sleeve 15 is deployed in a plate 10 and into a bone, the distal end of shank 60 will extend beyond the cortical bone/cancellous bone interface, so as to provide enhanced stabilization.

Thus, advancing screw 20 into sleeve 15 radially expands both the distal and proximal ends of sleeve 20, such that the sleeve is simultaneously secured to both the bone and the plate, as will hereinafter be discussed in further detail.

Bore 95 is preferably threaded so as to securely receive the shank of screw 20.

A radially-extending detent 105 is preferably formed in the side wall of counterbore 102, in order to receive a counterpart locking finger (see below) of screw 20, whereby to releasably lock screw 20 to sleeve 15, as will hereinafter be discussed in further detail.

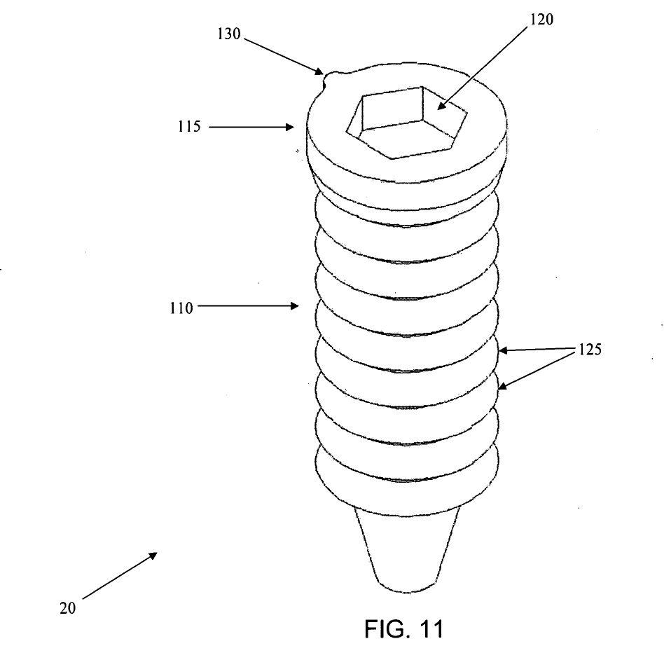

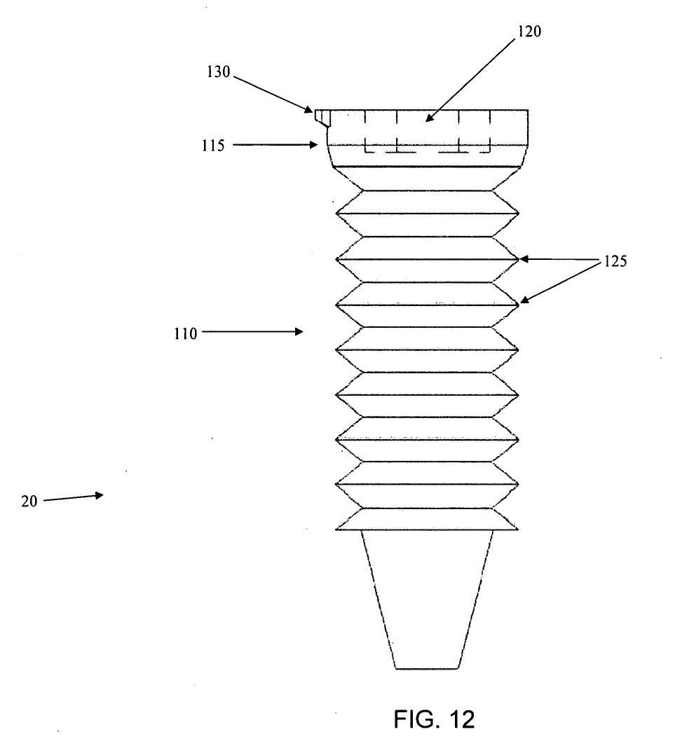

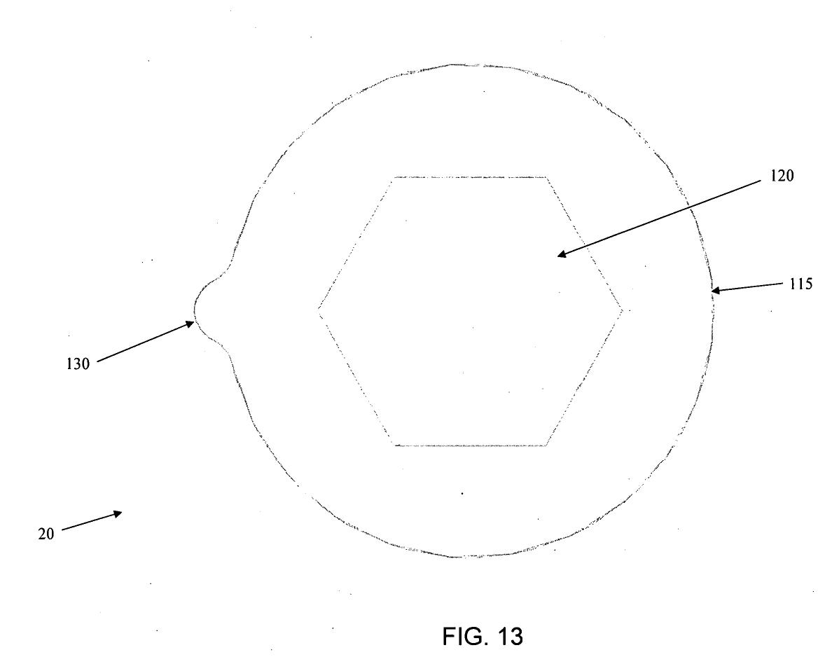

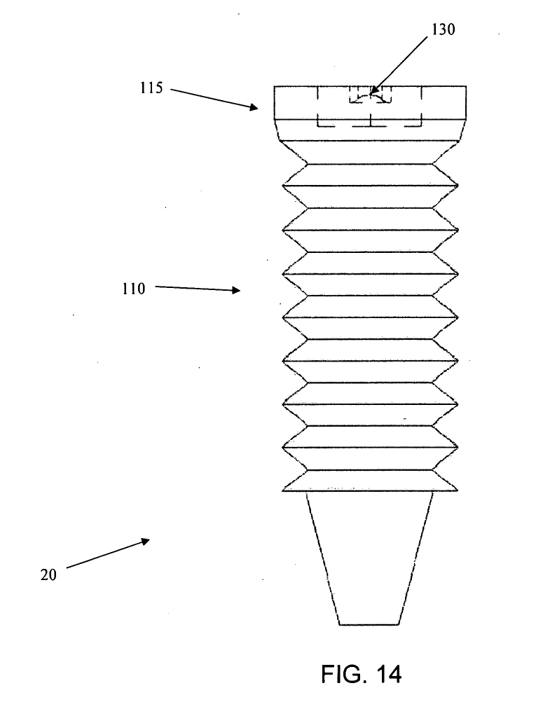

Screw 20 is shown in detail in FIGS. 11-14. Screw 20 generally comprises a shank 110, a head 115 and an opening 120 extending longitudinally into screw 20. Shank 110 comprises a thread 125 on its outer surface. As noted above, head 115 includes a radially-extending locking finger 130 for seating in the radially-extending detent 105 formed in sleeve 15, whereby to releasably lock screw 20 to sleeve 15, as will hereinafter be discussed in further detail. Opening 120 has a non-circular cross-section (e.g., hexagonal), in order that screw 20 can be rotatably driven by an appropriate driver. Preferably screw 20 is sized so that when it is seated within sleeve 15, the distal end of the screw projects out of the distal end of the sleeve (see FIG. 1).

Sleeve 15 and screw 20 can be used to secure a plate to bone. By way of example but not limitation, sleeve 15 and screw 20 can be used to secure plate 10 to a fractured bone so as to stabilize that bone. In this circumstance, plate 10 extends across the fracture line, with each end of the plate being secured to the bone using a sleeve/screw construction. Significantly, each sleeve/screw construction can be oriented at a different angle relative to plate 10, so as to better distribute load and/or apply a compressive force.

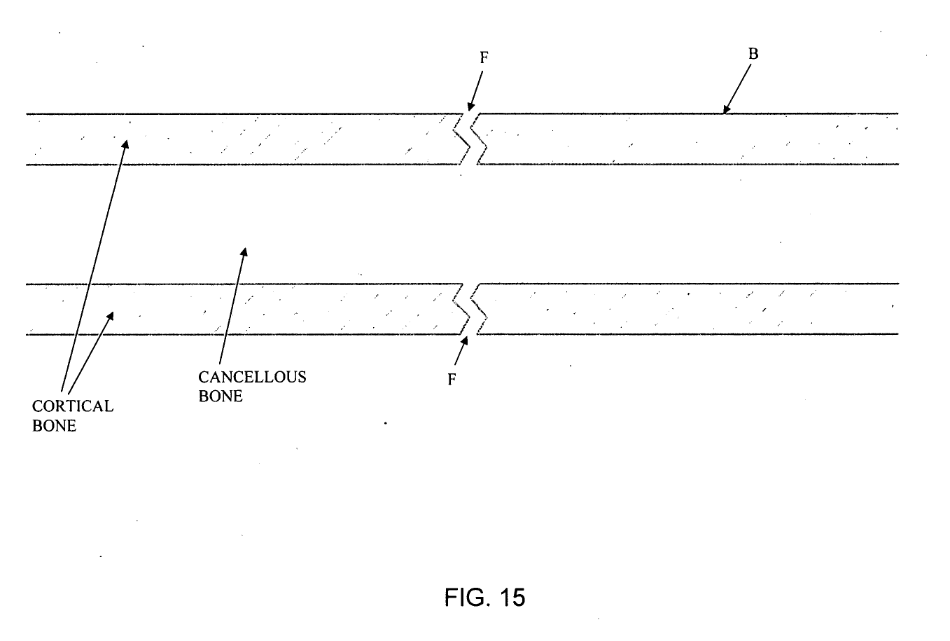

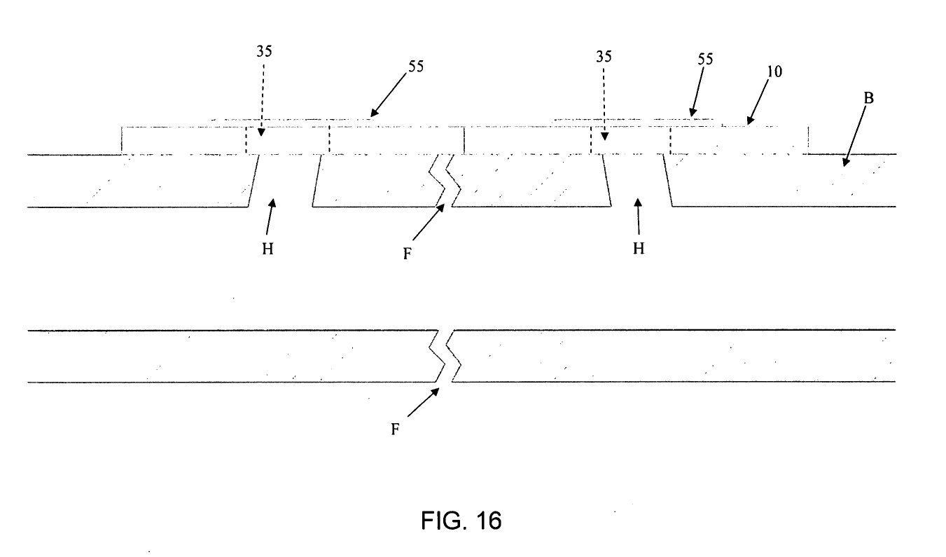

More particularly, and looking now at FIG. 15, there is shown a bone B having a fracture F. In order to stabilize fracture F, a plate may be secured to the bone on either side of fracture F. To this end, and looking now at FIG. 16, plate 10 is positioned against bone B, and then a bone hole H is drilled into the bone beneath each of the openings 35 which are to receive a sleeve/screw construction. This is done by passing a drill through opening 35 in plate 10 and into the bone. Due to the construction of plate 10 and sleeve 15, bone hole H can be set at any one of a number of different orientations relative to plate 10, e.g., bone hole H can extend at an acute angle relative to the plane of plate 10 (see, for example, FIG. 16) or bone hole H can extend at a right angle to the plane of plate 10 (not shown). This construction allows the surgeon to select the most desirable orientation for the bone hole, taking into account factors such as bone quality, force distribution, angle of approach, etc.

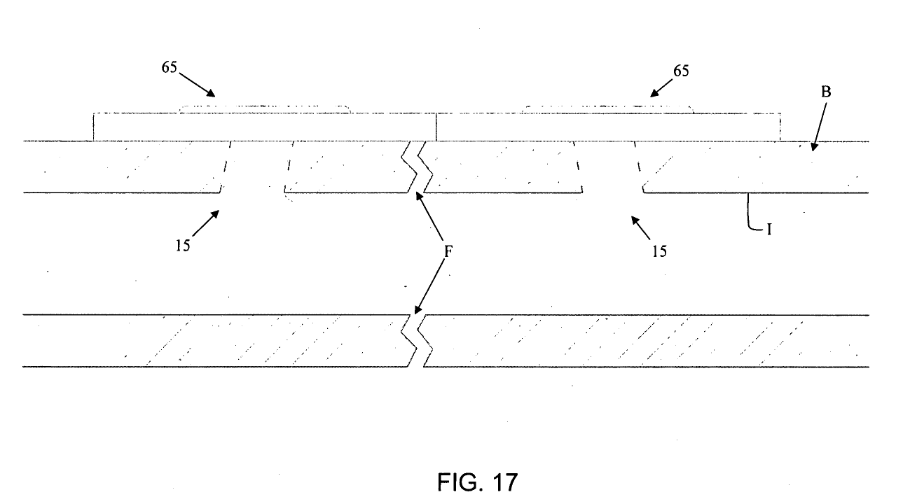

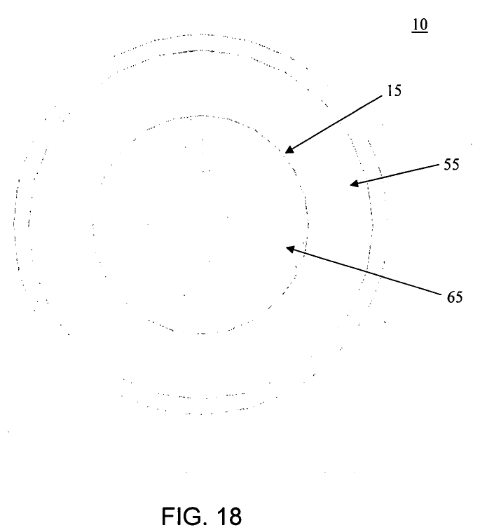

Once bone holes H have been drilled in bone B, sleeves 15 are advanced through plate openings 35 and into bone holes H (FIGS. 17 and 18). This is done by turning sleeve 15 with an appropriate rotational driver. Sleeve 15 is advanced until shank 60 is disposed in bone B and head 65 is seated in plate counterbore 45. At this point, sleeve 15 will serve to provide some degree of attachment of plate 10 to bone B, by virtue of the engagement of screw threads 75 with bone B and head 65 with counterbore 45.

As noted above, sleeve 15 is preferably sized so that, when sleeve 15 is deployed in a plate 10 and into bone B (FIG. 17), the distal end of shank 60 extends beyond the cortical bone/cancellous bone interface I, so as to provide enhanced stabilization, as will hereinafter be discussed in further detail.

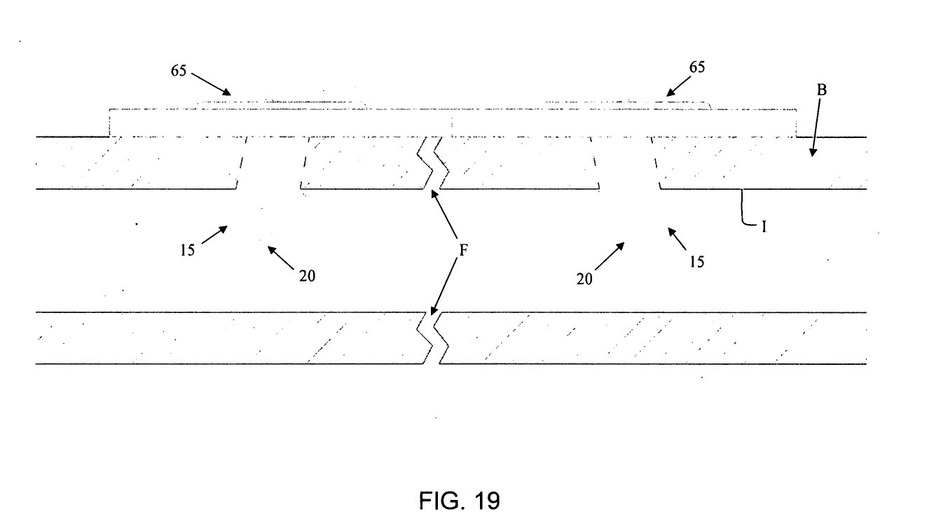

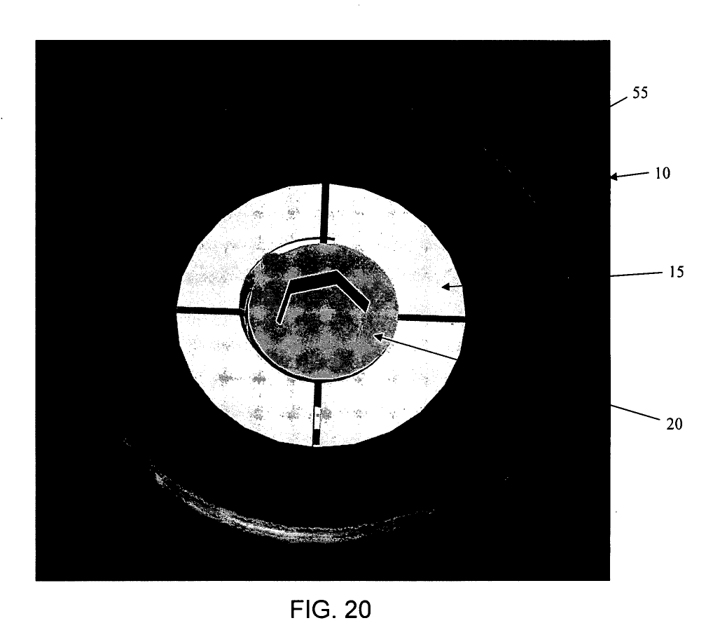

Next, screw 20 is advanced down opening 70 in sleeve 15 (FIGS. 19 and 20). As this occurs, sleeve 15 can be held against rotation using sleeve slots 85. The advancing screw 20 causes sleeve 15 to be radially expanded, so that the sleeve is simultaneously secured to both bone B and to plate 10. More particularly, the distal end of shank 60 of sleeve 15 is expanded so that the sleeve engages the cancellous portion of bone B, the proximal end of shank 60 of sleeve 15 engages the cortical portion of bone B, and head 65 of sleeve 15 engages plate 10. Significantly, sleeve 15 is sized so that the distal end of the sleeve mushrooms open beyond the cancellous bone/cortical bone interface I, making a tight securement between plate 10 and bone B.

Screw 20 is advanced until locking finger 130 seats in sleeve detent 105, thereby releasably locking the screw in position relative to the sleeve. Engagement of locking finger 130 in sleeve detent 105 also serves as an indicator, with tactile feedback, that the screw has been advanced to the proper extent (and not overtightened) relative to the sleeve.

Significantly, inasmuch as sleeve 15 opens laterally and presents a substantially larger profile than screw 20 alone, the disposition of the combination of sleeve and screw in the plate and the bone provides much better contact with the plate and the bone, thereby enhancing securement and shear resistance. This is particularly true since the distal end of sleeve 15 opens just beyond the cortical bone/cancellous bone interface I, so that plate 10 is secured to bone B under tension. In addition, since screw 20 is being advanced into sleeve 15 and not directly into the bone, there is little likelihood that the screw will lose its purchase and become a spinner. Furthermore, in the unlikely event that the screw should become a spinner, the situation can be easily rectified by removing screw 20 from sleeve 15 and removing sleeve 15 from the bone and plate 10. This leaves the host bone in condition for the procedure to be repeated with a new sleeve and/or a new screw, reusing the same bone hole.

### Additional Constructions of the Novel Fixation System

It is possible to modify the constructions described above without departing from the scope of the present invention.

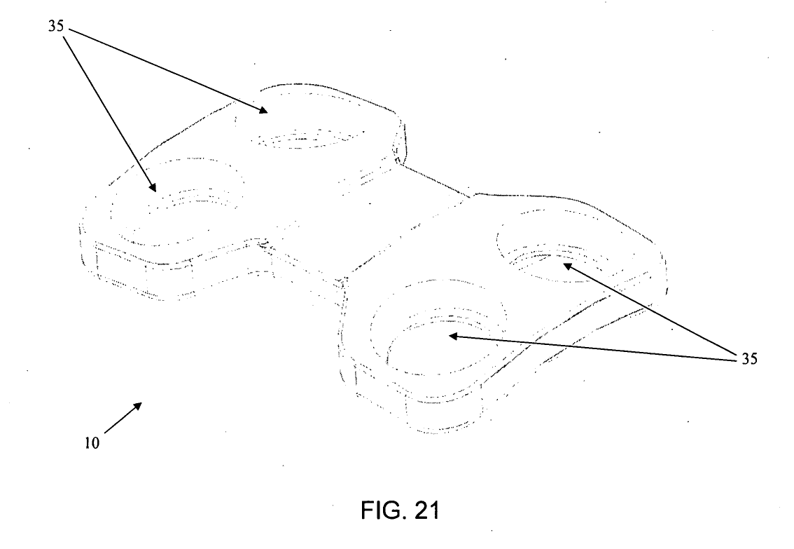

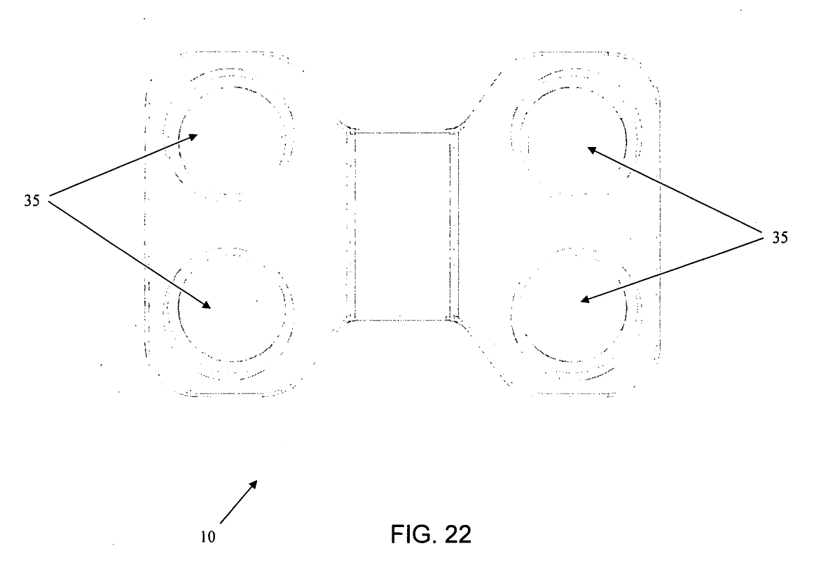

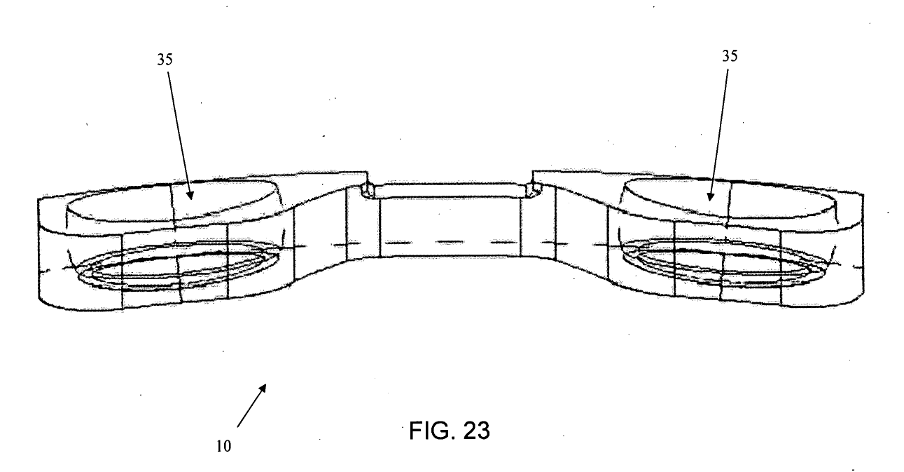

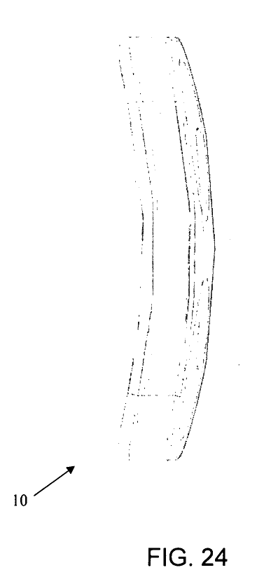

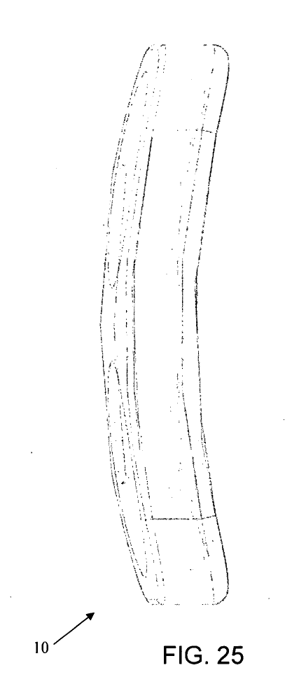

By way of example but not limitation, plate 10 might be formed with a non-rectangular and/or curved configuration, so as to seat more securely against a curved bone surface. See, for example, FIGS. 21-25, which show one such construction for plate 10.

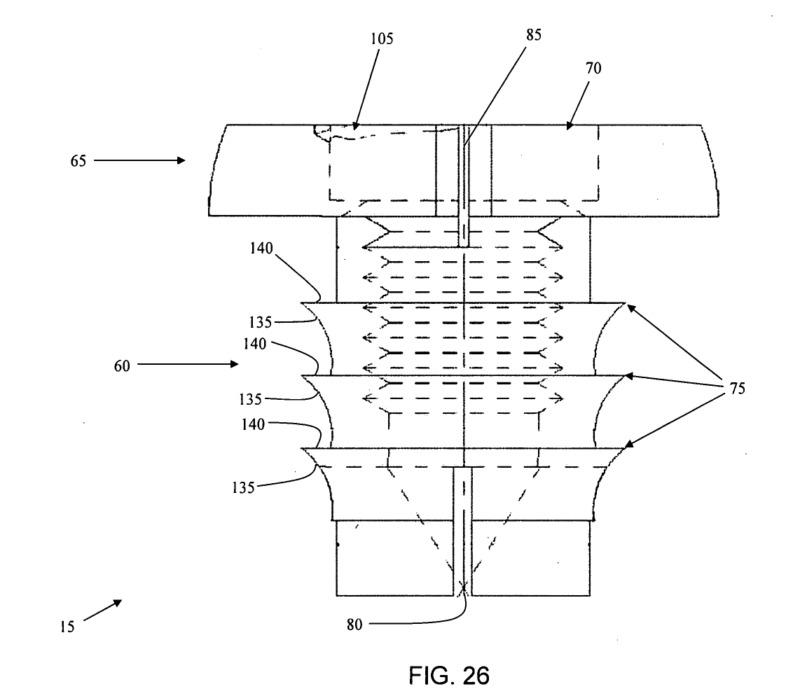

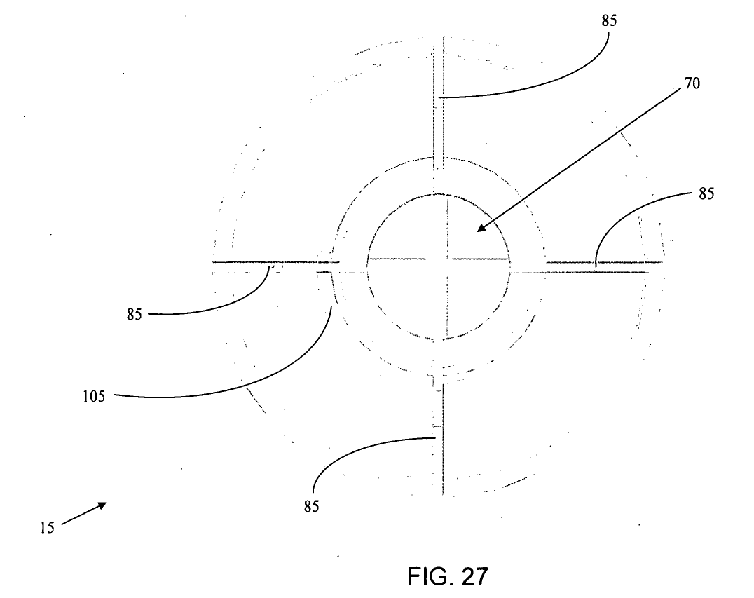

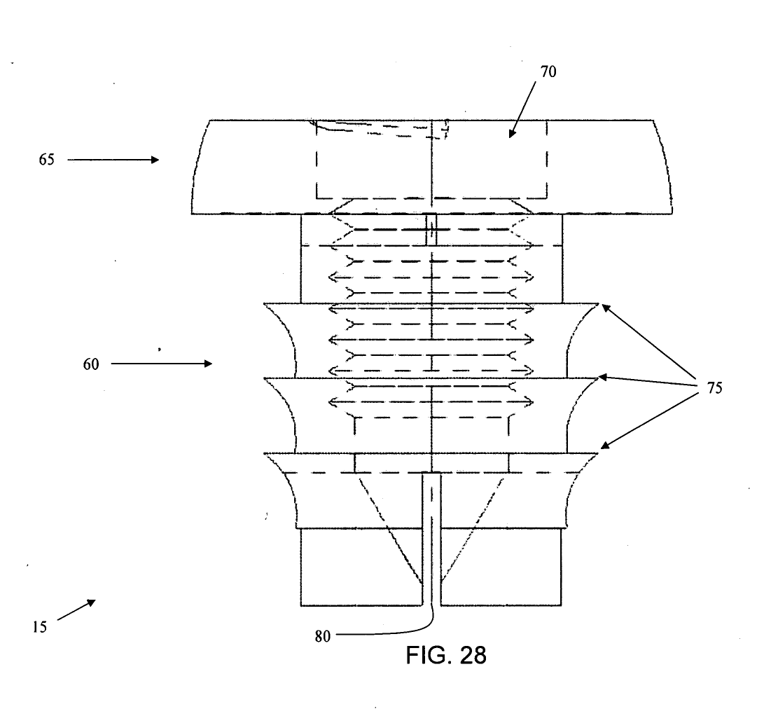

By way of further example but not limitation, sleeve 15 might be formed with ribs (or other lateral projections) 75 instead of a screw thread 75. See, for example, FIGS. 26-28, which show a sleeve 15 formed with ribs 75. In this case, sleeve 15 might be set with a mallet driver, etc., rather than with a rotational driver. Where sleeve 15 is formed with ribs 75, ribs 75 may be given a profile to facilitate insertion and impede withdrawal from the bone, e.g., sloped leading edges 135 and sharp rims 140.

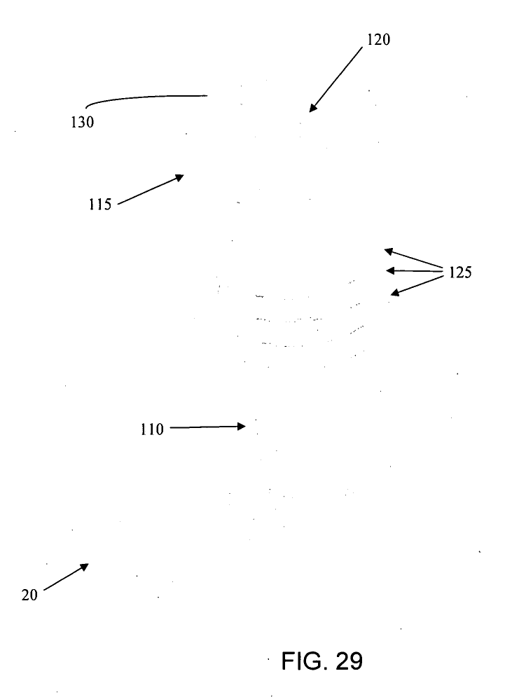

Also by way of example but not limitation, screw 20 may be sized to terminate within sleeve 15 rather than extend out the end of sleeve 15. Furthermore, screw thread 125 of screw 20 might be replaced by ribs (or other lateral projections) 125 for engaging the interior side wall of sleeve 15. See, for example, FIG. 29, which shows such a ribbed construction. In this case, or in other cases, the interior side wall of sleeve 15 might not be threaded. Additionally, screw 20 can be cannulated, so as to facilitate delivery over a guidewire.

Furthermore, sleeve 15 might be formed without a counterbore, and screw 20 might be formed without an enlarged head, in which case the screw would essentially constitute a threaded pin to be seated within a sleeve bore.

Additionally, the positions of detent 105 and finger 130 may be reversed, i.e., finger 130 may be formed on sleeve 15 and detent 105 may be formed on screw 20. Additionally, more than one detent and/or finger may be provided, e.g., the apparatus may comprise one finger and multiple detents.

Also, screw 20 and sleeve 15 may be pre-assembled (either at the time of manufacture or in the operating room) so as to constitute a single unit.

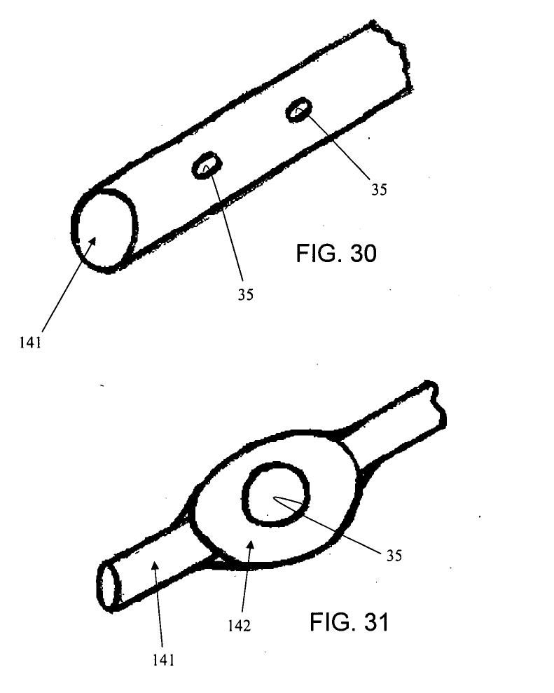

It should also be appreciated that the present invention may be used to secure a rod (or the like) to bone. By way of example but not limitation, the rod could be a spinal rod (or other surgical rod) used to stabilize a plurality of vertebral bodies relative to one another. In this case, a portion of the rod might be modified so as to be analogous to plate 10 (e.g., so as to provide one or more openings 35 through the rod for receiving sleeve 15 and screw 20). See FIG. 30, where a rod 141 is provided with one or more openings 35 therethrough. Where the rod has a relatively narrow diameter, and looking now at FIG. 31, a portion of rod 141 might be flattened and/or laterally expanded so as to provide an enlarged surface area 142 for receiving openings 35 to receive sleeve 15. However, where the rod has a relatively large diameter, openings 35 may be formed in the rod without requiring any flattening and/or lateral expansion of the rod.

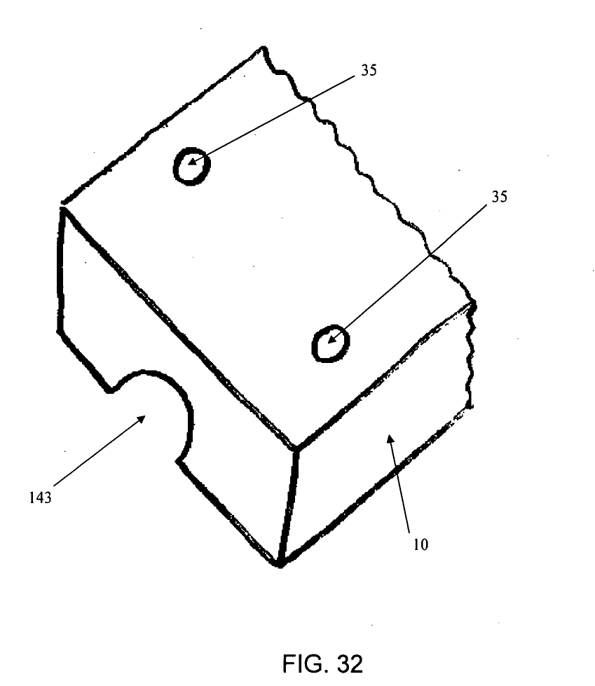

Alternatively, an adapter might be provided to secure the rod to bone. In this case, and looking now at FIG. 32, plate 10 could function as a rod mount, preferably with a groove 143 on the underside of the plate to capture the rod to the bone. In this case, it may be necessary to position openings 35 in plate 10 so that a sleeve 15 passing through openings 35 will pass alongside a rod captured in the groove. See FIG. 32.

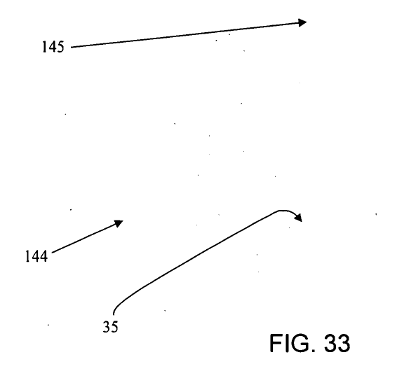

Additionally, the novel sleeve/screw construction can be used to secure a tulip-shaped mount to the bone, with the tulip-shaped mount being used to secure a rod to the bone. More particularly, and looking now at FIG. 33, a tulip-shaped mount 144 is shown, wherein the tulip-shaped mount has an opening 35 for securing the tulip-shaped mount to bone and a slot 145 for receiving a rod.

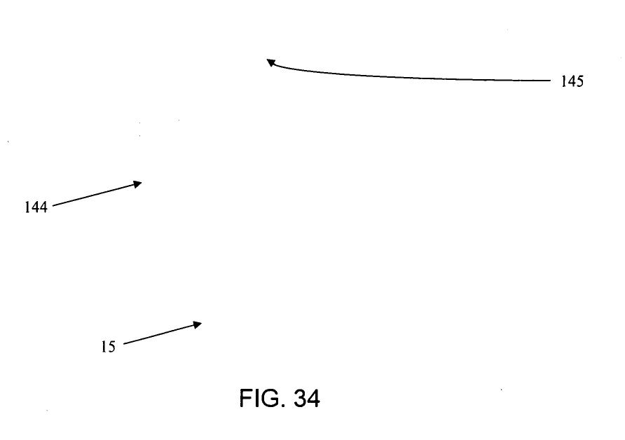

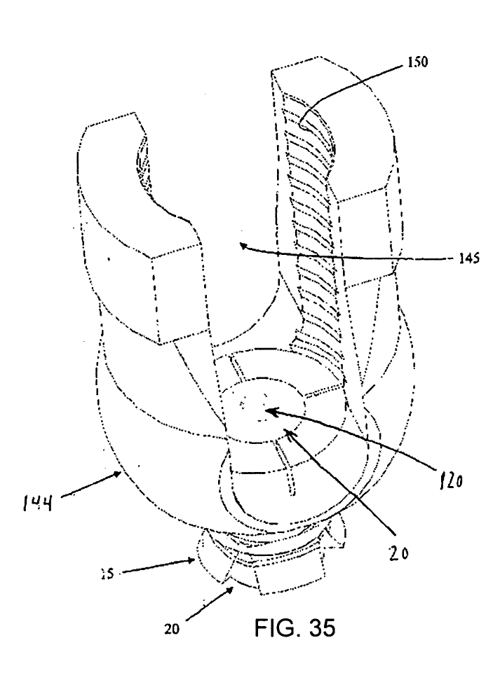

In use, tulip-shaped mount 144 is positioned alongside bone. A hole is drilled in the bone via opening 35 formed in tulip-shaped mount 144. Sleeve 15 is advanced through opening 35 (FIG. 34) and into the hole formed in the bone. Next, screw 20 is advanced through sleeve 15, causing sleeve 15 to be radially expanded, so that the sleeve is simultaneously secured to both the bone and to tulip-shaped mount 144 (see FIG. 35). With tulip-shaped mount 144 secured to the bone, a rod may be positioned in the slot 145 of tulip-shaped mount 144, whereby to stabilize the bone(s). If desired, tulip-shaped mount 144 may be provided with a threaded cap (not shown) which can be positioned superior to the rod using threads 150, so as to securely hold the rod in place within slot 145 of tulip-shaped mount 144.

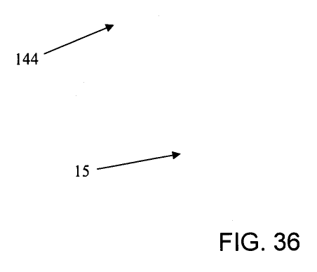

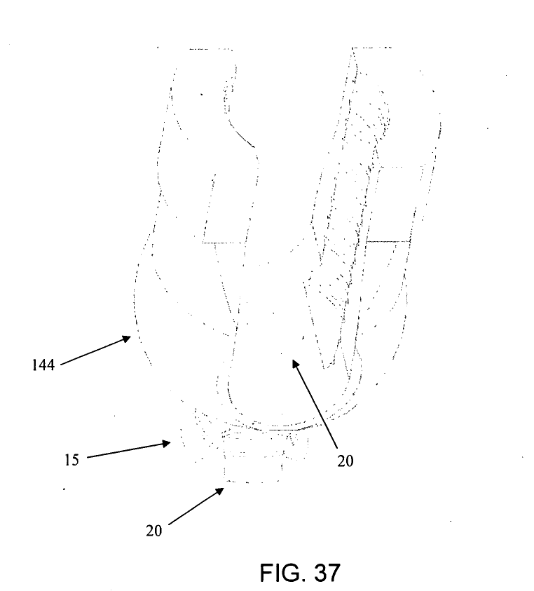

Looking next at FIGS. 36 and 37, it should also be appreciated that sleeve 15 can be formed integral with tulip-shaped mount 144.

### A Novel Anterior Cervical Plate (ACP) System for Stabilizing Bone

In many situations it may be necessary, or desirable, to stabilize bone. By way of example but not limitation, where a bone is fractured, it may be desirable to stabilize the bone with a bone plate which extends across the fracture line. By way of further example but not limitation, where two separate bones need to be secured together (e.g., in the case of a spinal fusion), it may be desirable to secure the bones to one another with a bone plate which extends from one bone to the other. In some cases, bridging or spacer material (e.g., allograft, autograft, biologic, etc.) may be placed as a graft between the two bones to stabilize and/or to enhance the fusion process of the two bones being secured together. Furthermore, in some situations (e.g., multi-level spinal surgery), it may be desirable to secure together more than two bones (e.g., in 3-level spinal surgery, it may be desirable to secure together three separate vertebral bodies). Again, bridging or spacer material may be placed as a graft between the individual bones.

In all of the foregoing situations, as well as in many other situations which are well known to those skilled in the art, a plate or plates generally need to be secured to bone in order to stabilize bone. Such securement is most commonly effected by using a surgical screw which passes through a hole in the plate and into the bone.

When using a surgical screw to secure a plate to bone, the plate is first aligned with the bone. Then a hole is drilled into the bone, by passing a drill through the pre-existing hole in the plate and into the bone. Next, the hole may be tapped. Then the surgical screw is screwed through the plate and into the hole in the bone.

Many different bone plates have been developed. In general, the configuration of these bone plates depends on their use, e.g., a fracture fixation plate may have one configuration, a spinal fusion plate may have another configuration, etc. Typically, the plate configuration seeks to balance anatomical configurations, anatomical loads, etc.

Over the past decade or so, anterior cervical fusion (ACF) has gained wide spread acceptance in the spinal community. In general, this procedure involves fusing together two (1-level) or more (multi-level) vertebral bodies. An anterior cervical plate (ACP) is commonly used to hold the vertebral bodies in position while bone fusion occurs.

Current ACPs all suffer from one or more disadvantages, including configurations which do not adequately accommodate anatomical limitations, designs which do not adequately stabilize anatomical loads, etc. Furthermore, current ACPs are not designed to accommodate subsequent surgeries where additional levels of fixation must be added. By way of example, current ACPs are not designed to facilitate converting a 1-level fixation to a 2-level fixation.

The present invention is intended to provide a new and improved ACP which improves upon the limitations of the prior art, including providing (i) improved anatomical accommodation, (ii) improved load stabilization, (iii) optional future level extensions, etc.

The present invention is intended to address the foregoing deficiencies of the prior art by providing a new and improved method and apparatus for stabilizing bone in general, and vertebral bodies in particular.

Among other things, the present invention provides a new and improved ACP system for stabilizing two or more cervical bodies.

In one preferred form of the present invention, the new ACP system comprises a plate which is to be attached to two adjacent cervical bodies, and attachment apparatus for attaching the ACP to the two cervical bodies. Preferably, the attachment apparatus comprise a screw and, in one preferred form of the invention, the attachment apparatus comprise a sleeve and screw combination, where the sleeve acts as an interface between (i) the bone and the screw, and (ii) the ACP and the screw, with the sleeve enhancing fixation. Among other things, the ACP is specifically configured to provide the option of adding future level extensions.

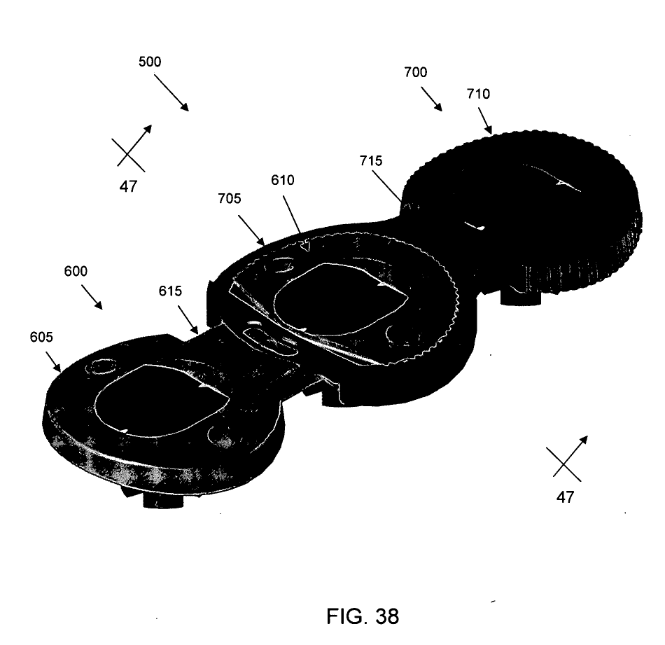

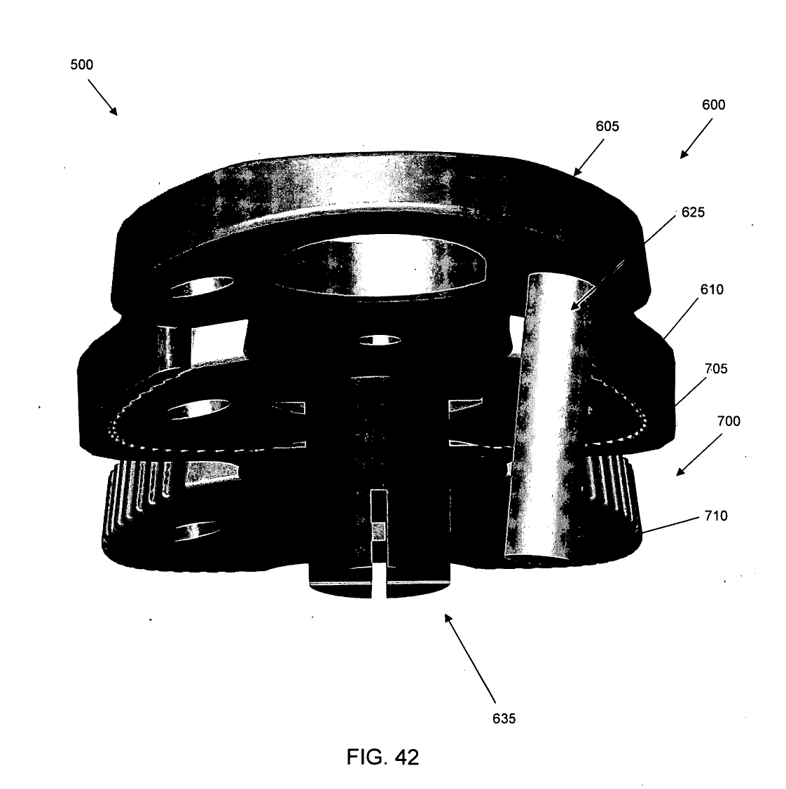

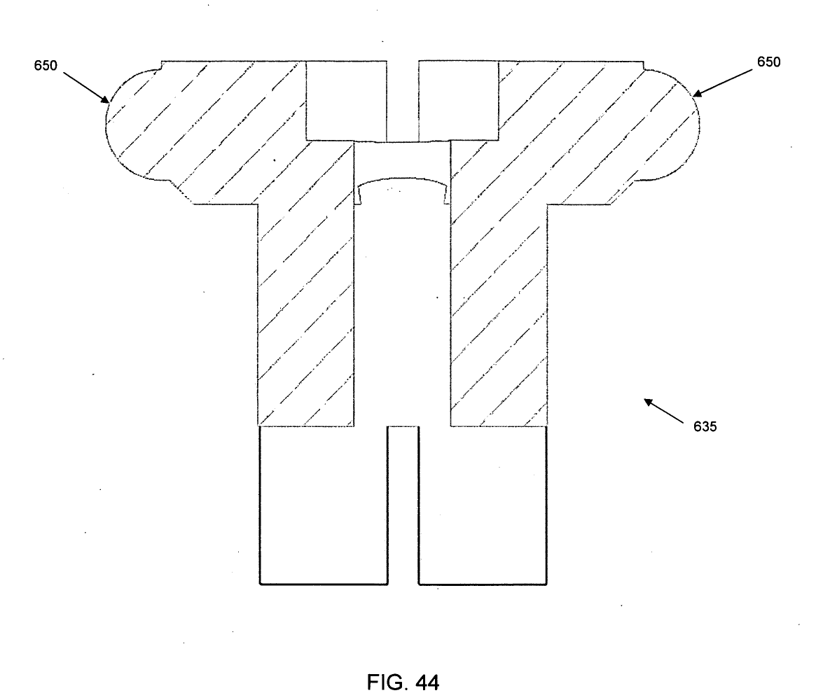

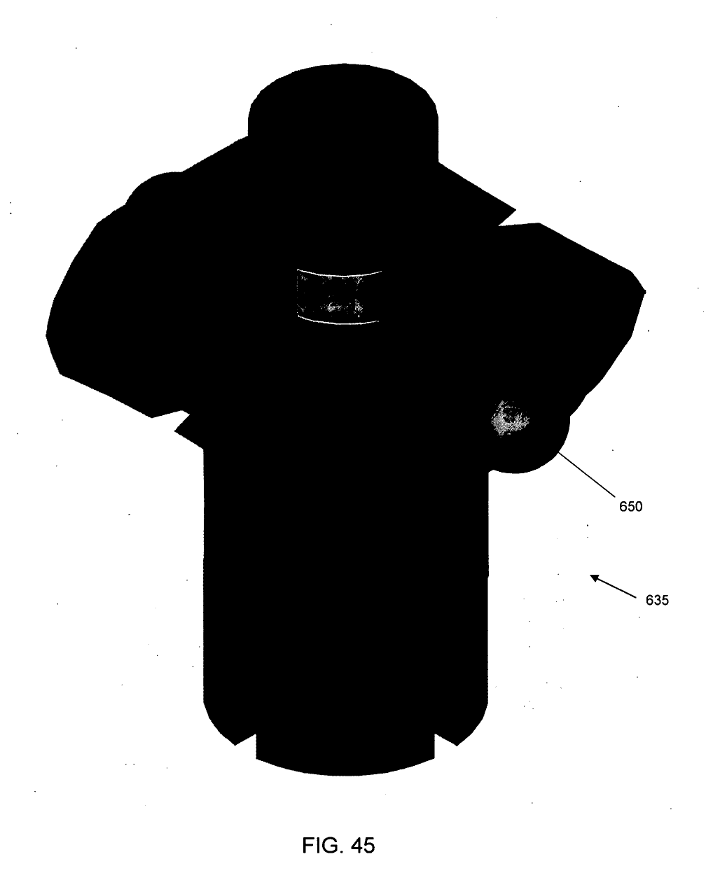

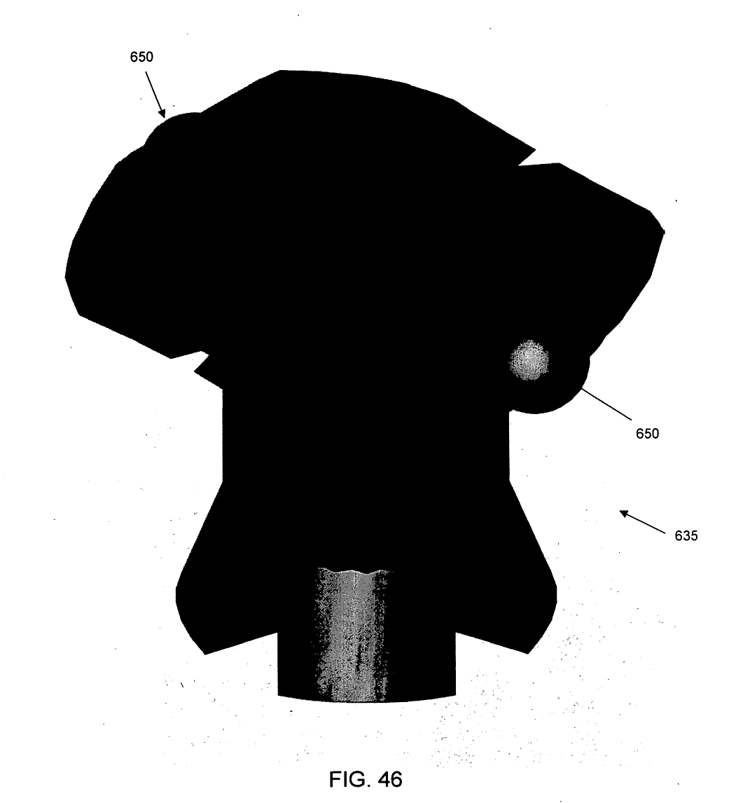

Looking now at FIGS. 38-46, there is shown a new and improved ACP system 500 for stabilizing two or more cervical bodies relative to one another. ACP system 500 generally comprises a primary ACP 600 for effecting a 1-level stabilization, and may further comprise one or more supplemental ACPs 700 for effecting subsequent 1-level stabilizations. Thus, for example, where a 1-level stabilization is to be initially established, and a further 1-level stabilization is to be thereafter established, ACP system 500 may comprise a primary ACP 600 and a secondary ACP 700, whereby to collectively establish the desired 2-level stabilization.

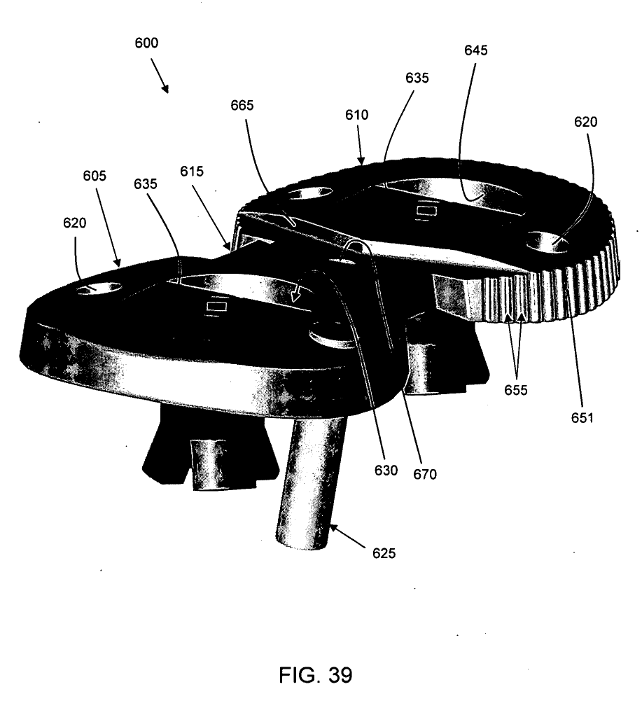

Looking now at FIG. 39, primary ACP 600 generally comprises a first, generally toroidal body 605, a second generally toroidal body 610, and a bridge 615 connecting first generally toroidal body 605 to second generally toroidal body 610. First toroidal body 605 and second toroidal body 610 each include (i) at least one opening 620 for receiving a pin 625 for initially tacking primary ACP 600 to the cervical bodies, and (ii) at least one opening 630 for receiving attachment apparatus 635 for thereafter securing primary ACP 600 to the cervical bodies.

Attachment apparatus 635 may comprise a spinal screw. More preferably, however, attachment apparatus 635 comprise a sleeve and screw combination of the sort discussed above (i.e., sleeve 15 and screw 20) and/or as disclosed in one or more of: (i) pending prior U.S. patent application Ser. No. 10/246,304, filed Sep. 18, 2002 by Barry T. Bickley for FIXATION AUGMENTATION DEVICE AND RELATED TECHNIQUES (Attorney's Docket No. BICK-002AUS); (ii) pending prior U.S. patent application Ser. No. 10/554,379, filed Oct. 25, 2005 by Barry T. Bickley et al. for FIXATION AUGMENTATION DEVICE AND RELATED TECHNIQUES (Attorney's Docket No. BICK-002BUS); (iii) pending prior U.S. patent application Ser. No. 12/148,845, filed Apr. 23, 2008 by Barry T. Bickley et al. for METHOD AND APPARATUS FOR SECURING AN OBJECT TO BONE (Attorney's Docket No. SIM-1); and/or (iv) pending prior U.S. patent application Ser. No. 12/156,495, filed Jun. 2, 2008 by Barry T. Bickley et al. for METHOD AND APPARATUS FOR SECURING AN OBJECT TO BONE AND/OR FOR STABILIZING BONE (Attorney's Docket No. SIM-2). These four patent applications are hereby incorporated herein by reference.

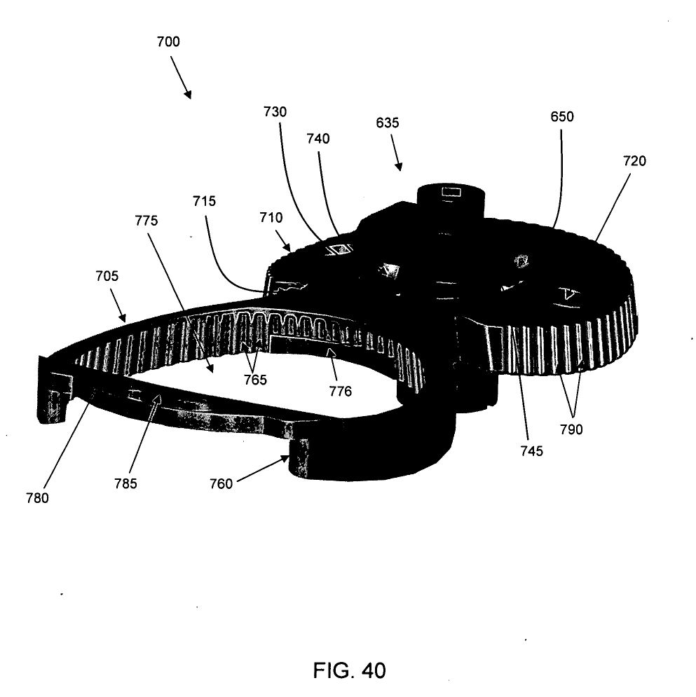

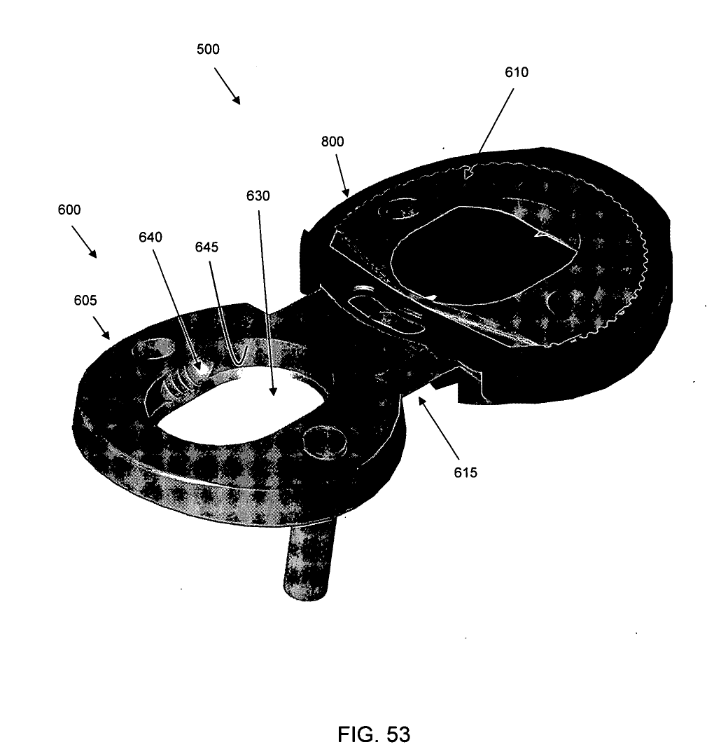

Preferably, primary ACP 600 includes recesses 640 (FIG. 53) formed in the sidewalls 645 which define openings 630. Recesses 640 help to releasably secure attachment apparatus 635 within openings 630, i.e., by receiving fingers 650 (FIG. 40) formed on the proximal end of attachment apparatus 635.

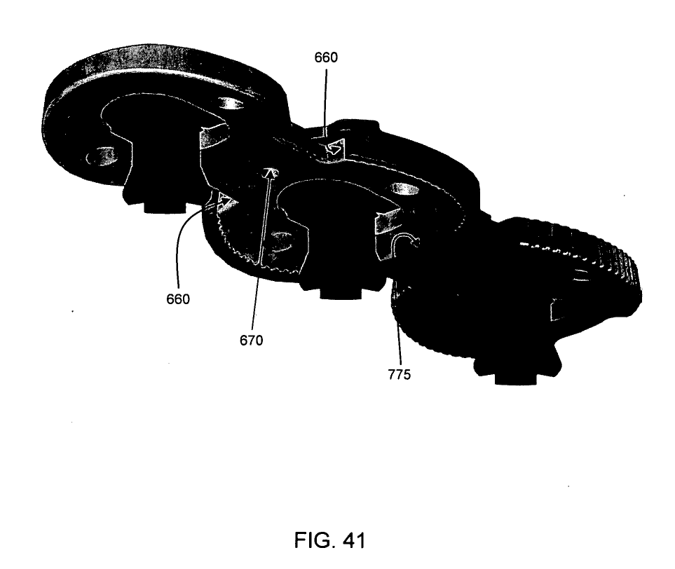

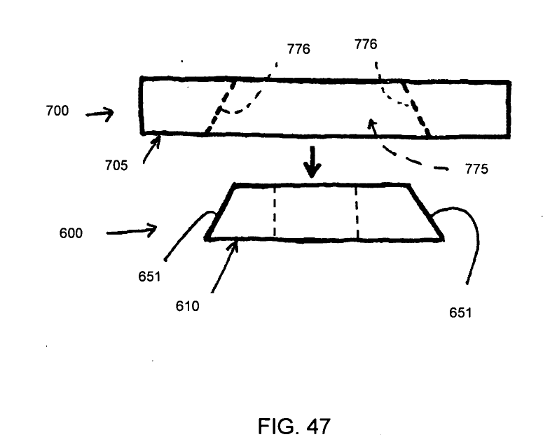

In order to facilitate the use of primary ACP 600 in conjunction with a supplemental ACP 700:
(i) the outer sidewall 651 forming the periphery of second toroidal body 610 is preferably formed with a taper (FIG. 47) in order to mate with a corresponding opening in supplemental ACP 700, as will hereinafter be discussed in further detail below;
(ii) primary ACP 600 preferably includes a plurality of teeth 655 extending along outer sidewall 651 of second toroidal body 610, in order to selectively lock primary ACP 600 to a supplemental ACP 700, as will hereinafter be discussed in further detail below;
(iii) primary ACP 600 is preferably cut back on its lateral edges, adjacent to where second toroidal body 610 meets bridge 615, i.e., at 660 (FIG. 41), in order to allow primary ACP 600 and a supplemental ACP 700 to assume a wide range of different positions, as will hereinafter be discussed in further detail below;
(iv) primary ACP 600 is preferably cut back on its proximal face, adjacent to where second toroidal body 610 meets bridge 615, i.e., at 665, in order to mate with a corresponding portion of a supplemental ACP 700, as will hereinafter be discussed in further detail below; and
(v) primary ACP 600 includes an opening 670 formed in its proximal face, to facilitate locking primary ACP 600 and a supplemental ACP 700, as will hereinafter be discussed in further detail.

Looking now at FIG. 40, supplemental ACP 700 generally comprises a first, generally toroidal body 705, a second generally toroidal body 710, and a bridge 715 connecting first generally toroidal body 705 to second generally toroidal body 710. Second toroidal body 710 includes (i) at least one opening 720 for receiving a pin (not shown) for initially tacking supplemental ACP 700 to a cervical body, and (ii) at least one opening 730 for receiving attachment apparatus 635 for thereafter securing supplemental ACP 700 to a cervical body.

Again, attachment apparatus 635 may comprise a spinal screw. More preferably, however, attachment apparatus 635 comprise a sleeve and screw combination of the sort discussed above (i.e., sleeve 15 and screw 20) and/or as disclosed in one or more of: (i) pending prior U.S. patent application Ser. No. 10/246,304; (ii) pending prior U.S. patent application Ser. No. 10/554,379; (iii) pending prior U.S. patent application Ser. No. 12/148,845; and/or pending prior U.S. patent application Ser. No. 12/156,495.

Preferably, supplemental ACP 700 includes recesses 740 formed in the sidewalls 745 which define opening 730. Recesses 740 help to releasably secure attachment apparatus 635 within openings 730, i.e., by receiving fingers 650 formed on the proximal end of attachment apparatus 635.

In order to facilitate use of supplemental ACP 700 with primary ACP 600:
(i) supplemental ACP 700 preferably includes a large opening 775 formed in its first toroidal body 705, and a sidewall 776 defining opening 775 is preferably formed with a taper (FIG. 47), in order to mate with the correspondingly-tapered second toroidal body 610 of primary ACP 600, as will hereinafter be discussed in further detail below;
(ii) supplemental ACP 700 preferably includes a plurality of teeth 765 lining at least a portion of opening 775, in order to selectively lock primary ACP 600 to a supplemental ACP 700, as will hereinafter be discussed in further detail below;
(iii) supplemental ACP 700 preferably has its first toroidal body 705 cut back adjacent to its free end, i.e., at 760, in order to allow primary ACP 600 and a supplemental ACP 700 to assume a wide range of different positions, as will hereinafter be discussed in further detail below; and
(iv) supplemental ACP 700 preferably includes strap 780 on its first toroidal body 705, with strap 780 including a slot 785, to facilitate locking primary ACP 600 and a supplemental ACP 700, as will hereinafter be discussed in further detail.

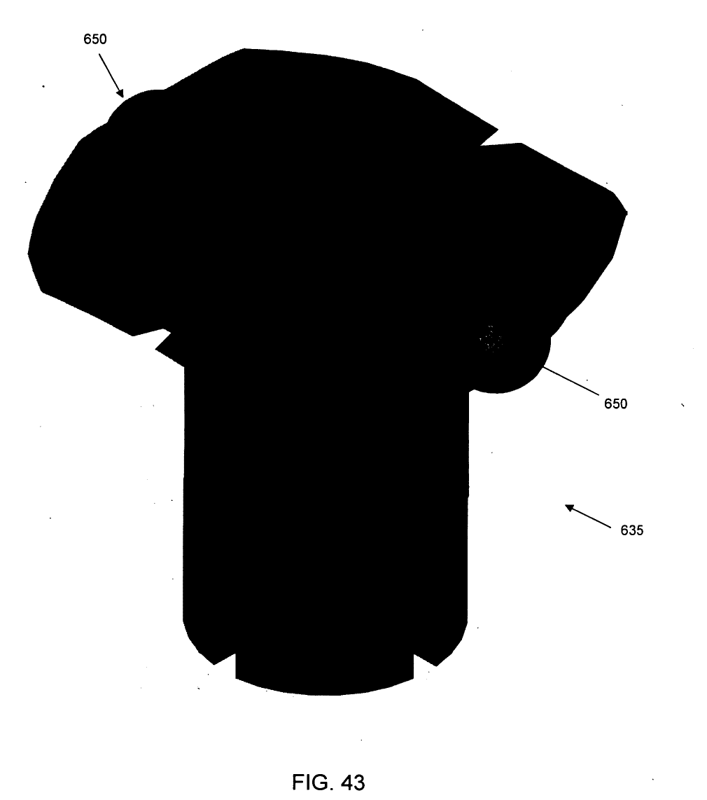

In use, primary ACP 600 is initially used to establish 1-level cervical stabilization. This is done by first positioning the two cervical bodies in the desired position, with or without bridging or spacer material (e.g., allograft, autograft, biologic, etc.) being placed as a graft between the two bones to stabilize and/or to enhance the fusion process of the two bones being secured together. Then primary ACP 600 is positioned against the two cervical bodies, with first toroidal body 605 of primary ACP 600 being positioned against one cervical body, and second toroidal body 610 of primary ACP 600 being positioned against a second cervical body. Primary ACP 600 is then pinned to the two bodies, i.e., using pins 625 extending through openings 620. Alternatively, primary ACP 600 may be pinned to one of the two bodies, the positioning of the two bodies may then be adjusted, and then primary ACP 600 pinned to the other of the two bodies. Thereafter, primary ACP 600 is secured to the two cervical bodies by passing attachment apparatus 635 through openings 630. By forming the head of attachment apparatus 635 with a hemispherical profile, and by forming the sidewalls of openings 630 with a corresponding arced profile, attachment apparatus 635 can be set at a range of angles “off the perpendicular” in order to accommodate various surgical considerations, e.g., patient anatomy, load distribution, etc. Furthermore, by forming the head of attachment apparatus 635 with a reduced profile (see FIGS. 38 and 43), attachment apparatus 635 will present a lower profile to the surrounding tissue if and when attachment apparatus 635 are set “off the perpendicular”.

In addition to the foregoing, by using attachment apparatus 635 in the form of a sleeve and screw combination of the sort discussed above (i.e., sleeve 15 and screw 20) and/or as disclosed in one or more of: (i) pending prior U.S. patent application Ser. No. 10/246,304; (ii) pending prior U.S. patent application Ser. No. 10/554,379; (iii) pending prior U.S. patent application Ser. No. 12/148,845; and/or (iv) pending prior U.S. patent application Ser. No. 12/156,495, a significant advantage is obtained. More particularly, by using attachment apparatus 635 of this type, the sleeve is effectively interposed between the screw and the ACP. Thus, it is the sleeve which is loaded by the ACP and therefore there is no transfer of motion forces directly onto the screw. As a result, there is a reduced tendency for the screw to back out over time.

If and when the 1-level stabilization of primary ACP 600 needs to be extended to a 2-level stabilization, a supplemental ACP 700 is used. More particularly, and looking still at the figures, first toroidal body 705 of supplemental ACP 700 is fit over second toroidal body 610 of primary ACP 600, with second toroidal body 610 of primary ACP 600 being received in large opening 775 (FIG. 41) of first toroidal body 705 of supplemental ACP 700. Seating of second toroidal body 610 of primary ACP 600 in large opening 775 of supplemental ACP 700 is facilitated by complementary tapered surfaces 651, 776 (FIG. 47). Furthermore, by forming primary ACP 600 with surfaces 651 which taper inwardly as they move away from the bone, and by forming supplemental ACP 700 with surfaces 776 which taper outwardly as they move toward the bone, fitting supplemental ACP 700 over primary ACP 600 will help clear away any tissue which may have grown over the primary ACP while it has been implanted (e.g., in a revision situation). As second toroidal body 610 of primary ACP 600 is received in large opening 775 (FIG. 41) of first toroidal body 705 of supplemental ACP 700, teeth 655 of primary ACP 600 engage with teeth 765 of supplemental ACP 700 so as to fix the two bodies relative to one another, with strap 780 of supplemental ACP 700 overlying bridge 615 of primary ACP 600. Then a screw (not shown) is passed through slot 785 in bridge 780 (FIG. 40) and into opening 670 in bridge 615 (FIG. 39), whereby to lock primary ACP 600 and supplemental ACP 700 into position relative to one another. Thereafter, supplemental ACP 700 is secured to the third cervical body by passing attachment apparatus 635 through opening 730. By forming the head of attachment apparatus 635 with a hemispherical profile, and by forming the sidewalls of openings 730 with a corresponding arced profile, attachment apparatus 635 can be set at a range of angles “off the perpendicular” in order to accommodate various surgical considerations, e.g., patient anatomy, load distribution, etc. Furthermore, by forming the head of attachment apparatus 635 with a reduced profile (see FIGS. 38 and 43), attachment apparatus 635 will present a lower profile to the surrounding tissue if and when attachment apparatus 635 are set “off the perpendicular”.

In addition to the foregoing, by using attachment apparatus 635 in the form of a sleeve and screw combination of the sort discussed above (i.e., sleeve 15 and screw 20) and/or as disclosed in one or more of: (i) pending prior U.S. patent application Ser. No. 10/246,304; (ii) pending prior U.S. patent application Ser. No. 10/554,379; (iii) pending prior U.S. patent application Ser. No. 12/148,845 and/or (iv) pending prior U.S. patent application Ser No. 12/156,495, a significant advantage is obtained. More particularly, by using attachment apparatus 635 of this type, the sleeve is effectively interposed between the screw and the ACP. Thus, it is the sleeve which is loaded by the ACP and therefore there is no transfer of motion forces directly onto the screw. As a result, there is a reduced tendency for the screw to back out over time.

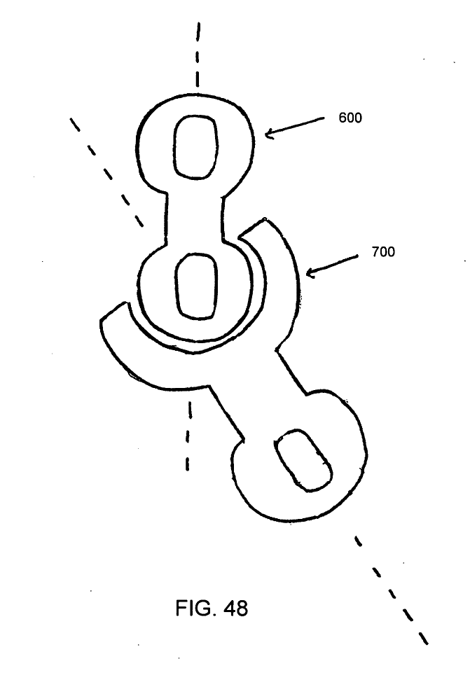

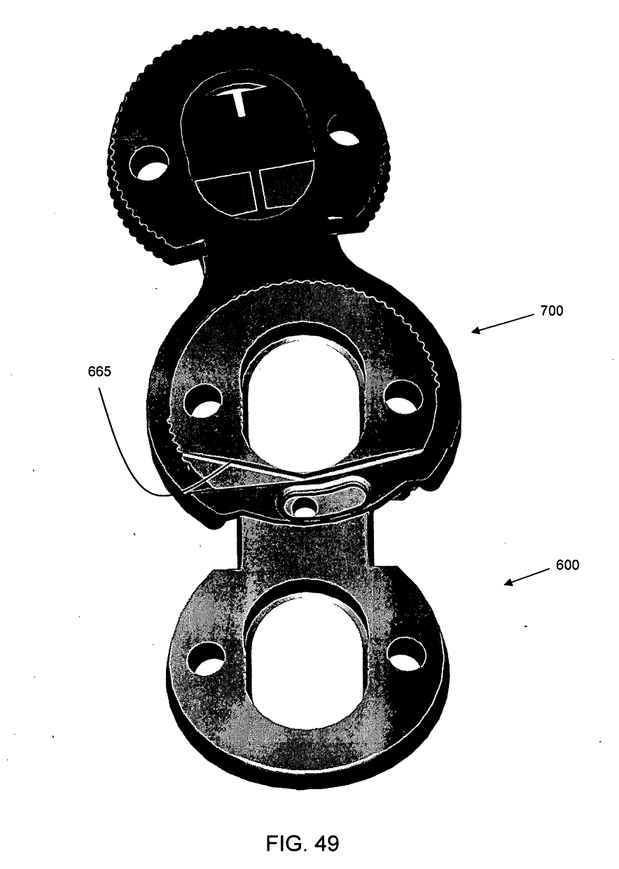

Due to the construction of primary ACP 600 and supplemental ACP 700, the primary ACP and the supplemental ACP can be aligned in a variety of orientations, i.e., on-axis (FIG. 38) or off-axis (FIGS. 48 and 49) before being secured. In essence, supplemental ACP 700 can be “dialed around” primary ACP 600, according to the particular anatomical situation encountered by the surgeon. This can be particularly helpful in revision cases, since the surgeon does not need to remove a mis-aligned primary ACP 600 in order to get proper alignment of a supplemental ACP 700.

If further levels of stabilization are required, additional supplemental ACPs 700 can be added in a serial fashion. To this end, second toroidal body 710 of supplemental ACP 700 includes teeth 790 for mating with teeth 765 of an immediately-proceeding supplemental ACP 700. Again, each incremental supplemental ACP 700 may be set on-axis or off-axis from its immediately-preceding ACP, as dictated by the existing position of the immediately-preceding ACP and by the patient anatomy being encountered.

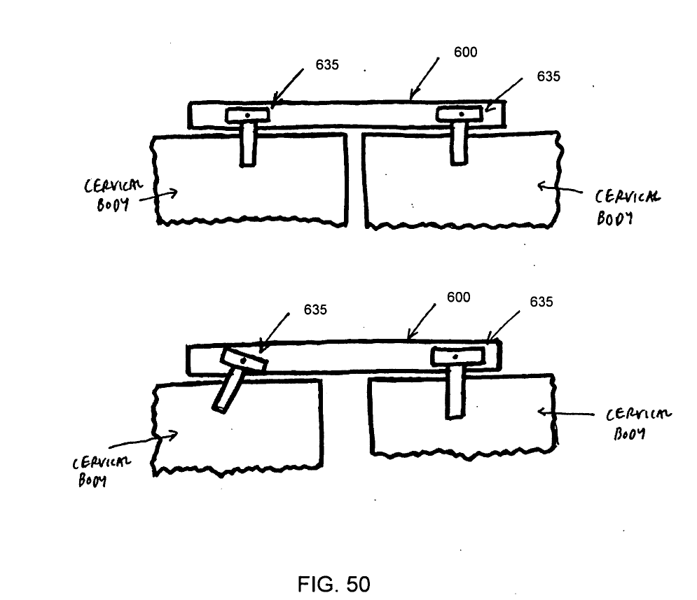

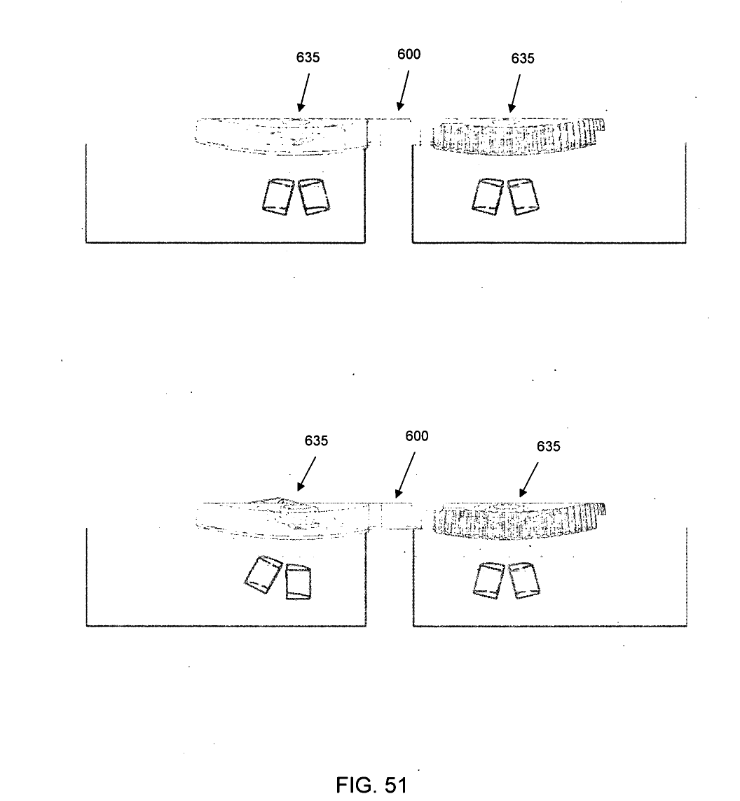

Among other things, it should be appreciated that when attachment apparatus 635 include receiving fingers 650 (FIG. 40), and when primary ACP 600 and supplemental ACP 700 include recesses 640, 740, attachment apparatus 635 are able to pivot relative to primary ACP 600 and supplemental ACP 700. This construction permits primary ACP 600 and/or supplemental ACP 700 to pivot relative to attachment apparatus 635 (and hence pivot relative to the cervical bodies receiving the distal ends of attachment apparatus 635), without permitting longitudinal and/or lateral translation of primary ACP 600 and/or supplemental ACP 700 relative to attachment apparatus 635 (and hence the cervical bodies receiving the distal ends of attachment apparatus 635). See FIGS. 50 and 51.

### Alternative Constructions of the Novel Anterior Cervical Plate (ACP)

If desired, primary ACP 600 may have more than one opening 630 per level, and/or supplemental ACP 700 may have more than one opening 730 per level. Furthermore, primary ACP 600 may extend for more than two levels, and/or supplemental ACP 700 may extend for more than two levels.

Furthermore, openings 630 and/or openings 730 may have a round or oval shape. The oval shape is generally preferred, since it provides an anti-rotation feature when attachment apparatus 635 comprise a sleeve and screw combination. Furthermore, the oval shape provides some opportunity for the attachment apparatus 635 to slide within the opening.

In addition to the foregoing, recesses 640 and 740 can comprise a hemisphere or an elongated slot. Where recesses 640 and 740 comprise an elongated slot, the slot can itself provide several seats to accommodate a range of engagements. By way of example but not limitation, the slot can comprise a plurality of detents spaced along the length of the slot for selectively seating fingers 650, whereby to permit adjustable engagement of attachment apparatus 635 to primary ACP 600 and supplemental ACP 700.

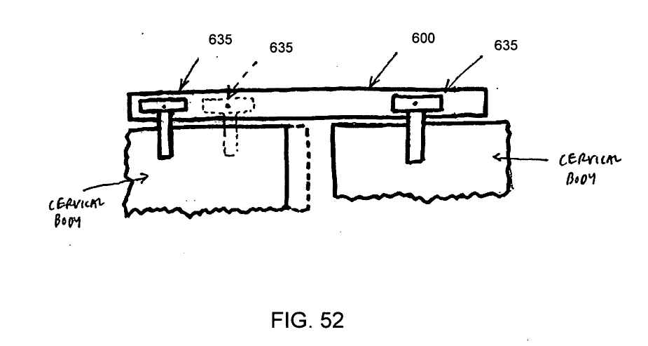

By forming openings 630, 730 with an oval shape, and by forming recesses 640, 740 in a slot configuration with several seats, dynamic fixation can be effected. More particularly, the foregoing construction permits primary ACP 600 and/or supplemental ACP 700 to translate longitudinally relative to attachment apparatus 635 (and hence translate longitudinally relative to the cervical bodies receiving the distal ends of attachment apparatus 635), without permitting lateral translation of primary ACP 600 and/or supplemental ACP 700 relative to attachment apparatus 635 (and hence the cervical bodies receiving the distal ends of attachment apparatus 635). See FIG. 52.

It should also be appreciated that teeth 655 of primary ACP 600, teeth 765 of supplemental ACP 700, and teeth 790 of supplemental ACP 700 may all be replaced with facet structures. These facet structures may be configured so as to provide fast and simple alignment and assembly of adjoining ACPs.

### Protective Collars

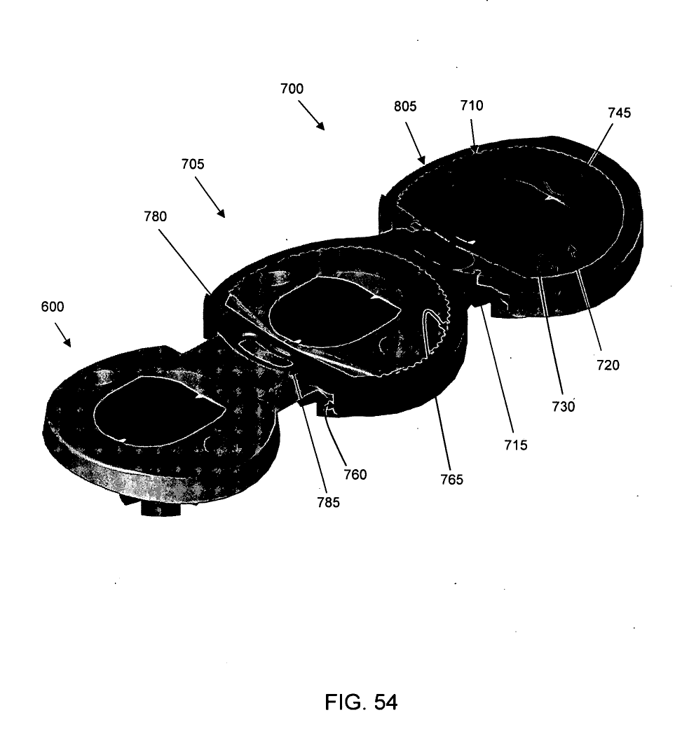

Primary ACP 600 and/or supplemental ACP 700 may be provided with a protective collar so as to minimize tissue ingrowth about second generally toroidal body 610 and/or second generally toroidal body 710, respectively. See, for example, FIG. 53, which shows a protective collar 800 set about second generally toroidal body 610, and FIG. 54, which shows a protective collar 805 set about second generally toroidal body 710.

Protective collars 800 and/or 805 are preferably pre-applied to primary ACP 600 and/or supplemental ACP 700, respectively, prior to deployment of the ACP into the body, although the protective collars may also be applied after an ACP has been deployed in the body.

If primary ACP 600 and/or supplemental ACP 700 is equipped with a protective collar, and if an additional level of fixation is to be added (i.e., if a supplemental ACP 700 is to be added to the ACP structure(s) already in place), that protective collar is removed before the supplemental ACP is deployed, so that the supplemental ACP can be fixed to the ACP structure(s) already in place.

### Alternative Fixation System Comprising a Plate, a Sleeve and a Screw for Securing an Object to Bone and/or for Stabilizing Bone

It is also possible to use the present invention to mount a different type of plate to bone, and/or to modify the construction of sleeves 15 and/or screws 20 without departing from the scope of the present invention.

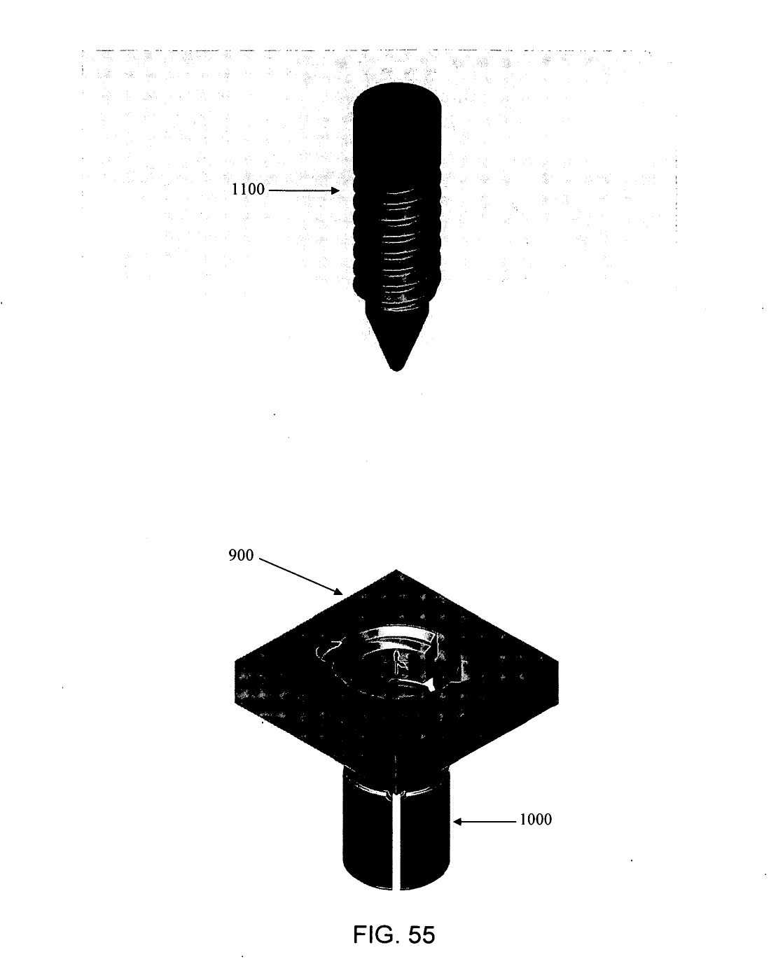

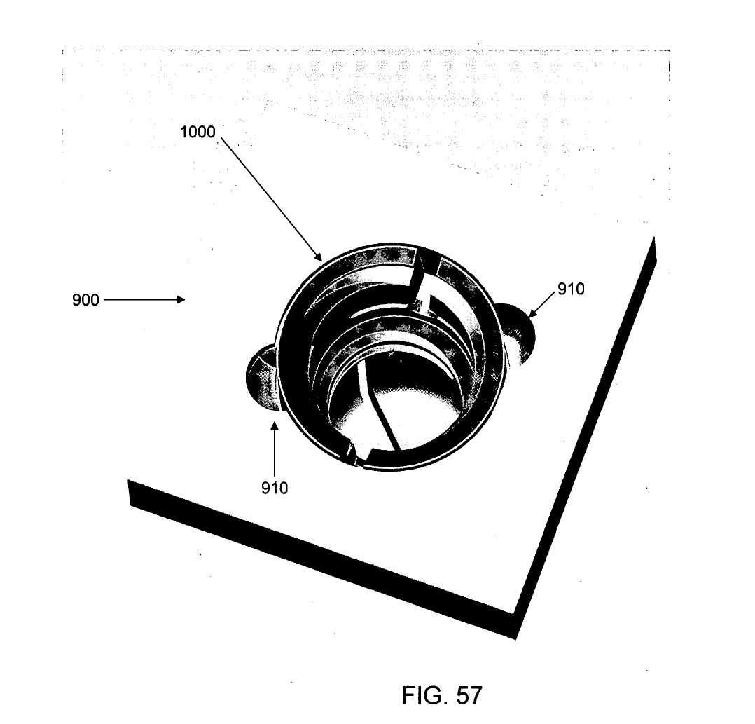

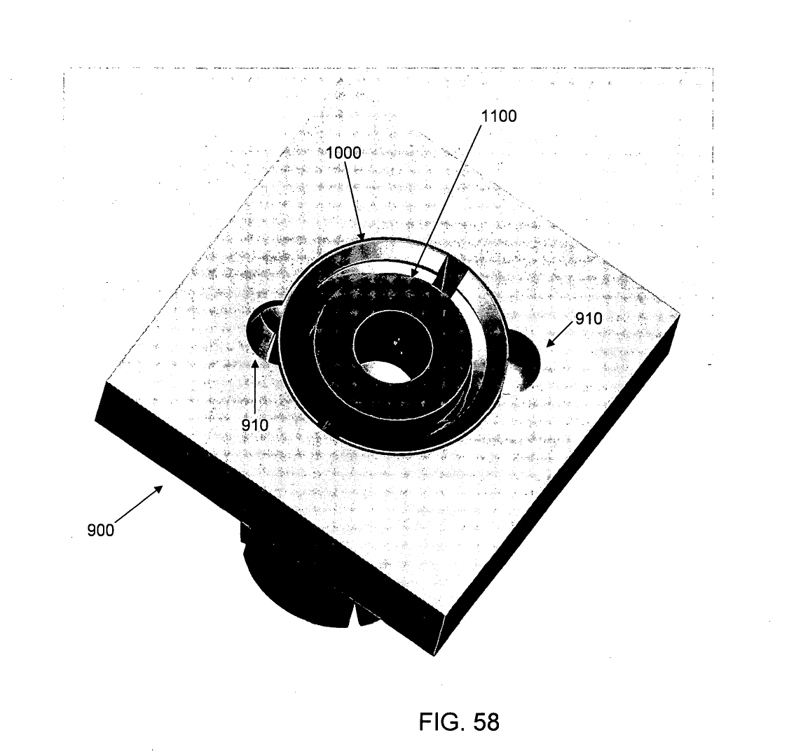

More particularly, and looking now at FIGS. 55-58, there is shown a fixation system comprising a plate 900 which is to be secured to bone, a sleeve 1000 and a screw 1100, with sleeve 1000 and screw 1100 being adapted to secure plate 900 to bone.

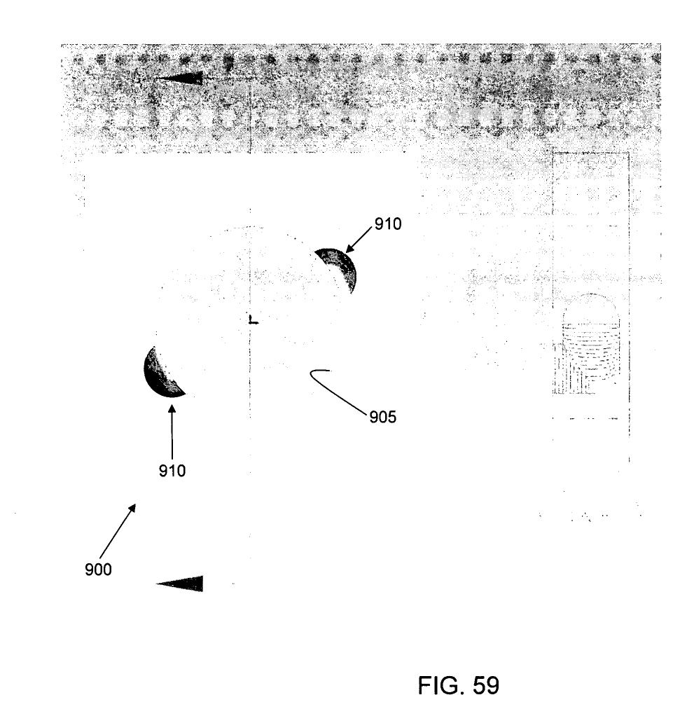

Plate 900 is shown in detail in FIGS. 55-59. Plate 900 generally comprises a bore 905 and a pair of “bayonet mounts” 910. Bayonet mounts 910 generally comprise an opening 915 which communicates with an undercut 920.

Depending on the intended use of plate 900, more than one opening 905 may be provided. By way of example but not limitation, where plate 900 is intended to be used as a fracture fixation plate or as a spinal fusion plate, at least one (and preferably two or more) openings 905 are formed in plate 900 on either side of the bone separation line (e.g., the fracture line, the vertebral body abutment lines, etc.), such that plate 900 can be secured to bone on each side of the bone separation line. By way of further example but not limitation, where plate 900 is intended to be used to secure soft tissue to bone, plate 900 might include only one opening 905.

Additionally, it should also be appreciated that additional design features may be present on plate 900, depending on the intended use for plate 900. By way of example but not limitation, in one form of the invention, plate 900 might comprise a primary ACP 600 or a secondary ACP 700.

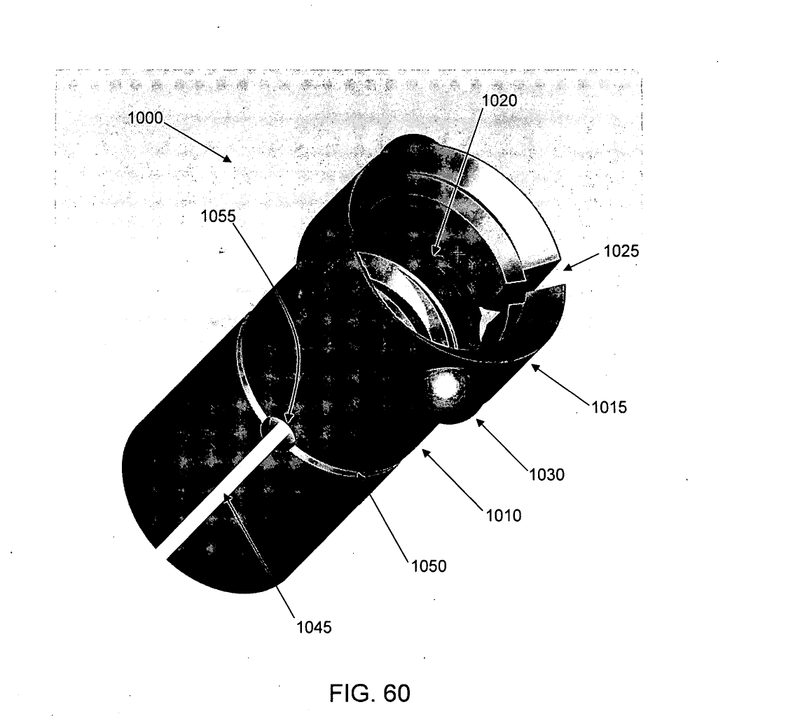

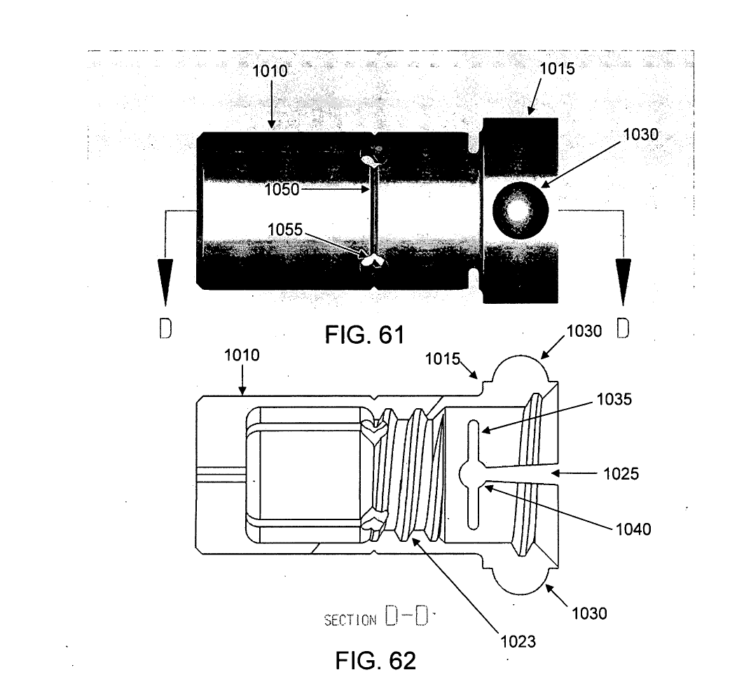

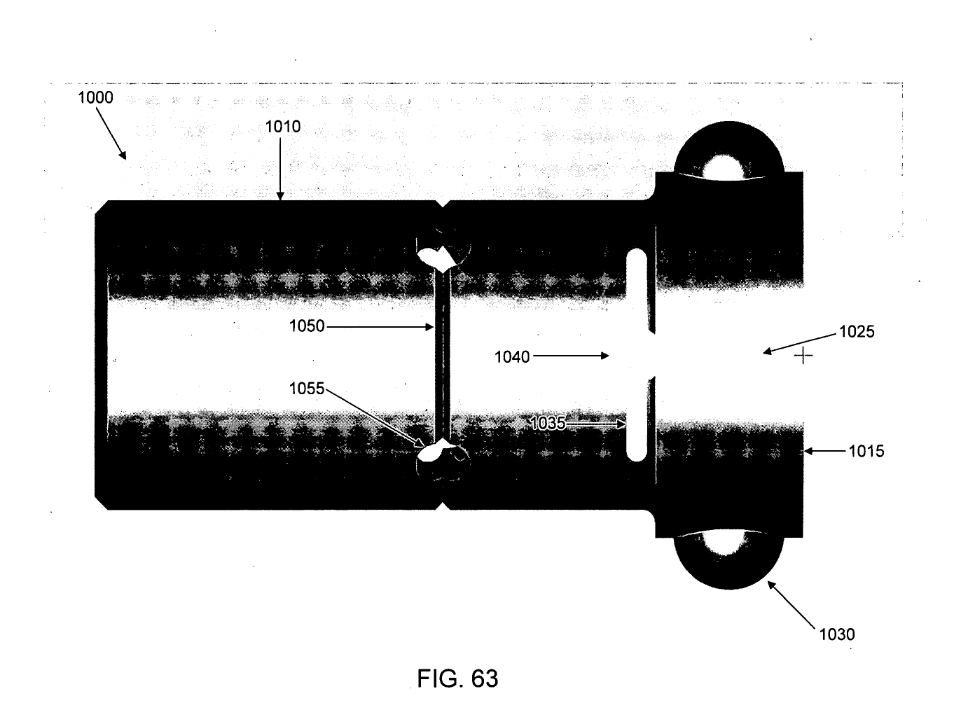

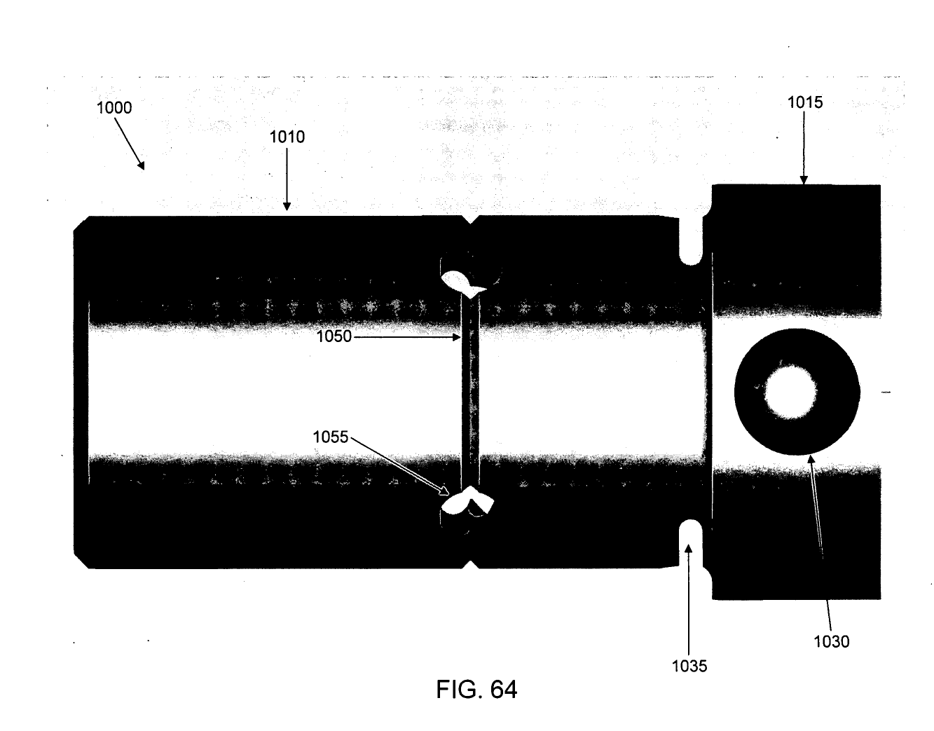

Sleeve 1000 is shown in detail in FIGS. 55-58 and 60-64. Sleeve 1000 generally comprises a body 1010, a head 1015 and a bore 1020 extending through head 1015 and into body 1010.

Body 1010 comprises threads 1023 (FIG. 62) formed within bore 1020. Threads 1023 are preferably configured to facilitate the gripping entry of screw 1100 into sleeve 1000 when screw 1100 is turned into sleeve 1000.

Head 1015 comprises at least one slit 1025 which opens at the proximal end of head 1015 and extends distally. Preferably, slit 1025 communicates with a slot 1035, wherein slot 1035 extends at a right angle to slit 1025. More particularly, slit 1025 preferably extends longitudinally along head 1015, and slot 1035 extends substantially circumferentially along body 1010. Preferably, an enlarged opening 1040 is formed at the intersection of slit 1025 and slot 1035. Enlarged opening 1040 acts as a stress relief between slit 1025 and slot 1035. The provision of slit 1025 and slot 1035 enhance the flexibility of head 1015 relative to body 1010 when screw 1100 is advanced into sleeve 1000 so as to expand sleeve 1000, as will hereinafter be discussed in further detail.

Head 1015 further comprises a pair of laterally extending pivots 1030. Laterally extending pivots 1030 are received in mounts 910 of plate 900, as will hereinafter be discussed in further detail.

Preferably, body 1010 also comprises at least one slit 1045 which opens on the distal end of body 1010 and extends proximally. Slits 1045 intersect a circumferential surface groove 1050 at an enlarged opening 1055. Slits 1045, circumferential surface groove 1050 and opening 1055 allow the distal end of body 1010 to expand against the bone when screw 1100 is received within sleeve 1000. To this end, circumferential surface groove 1050 acts as a stress relief as body 1010 expands radially against the bone.

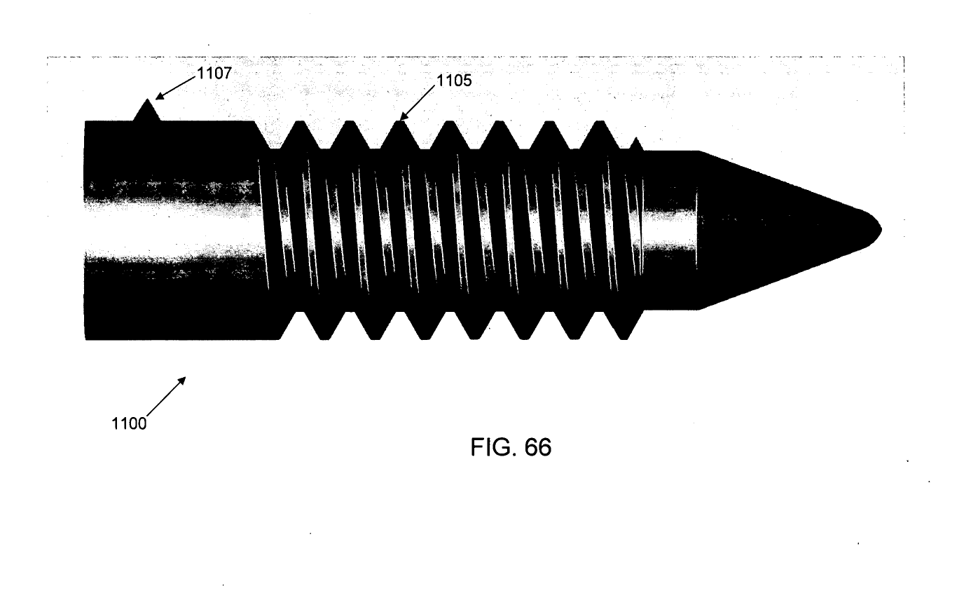

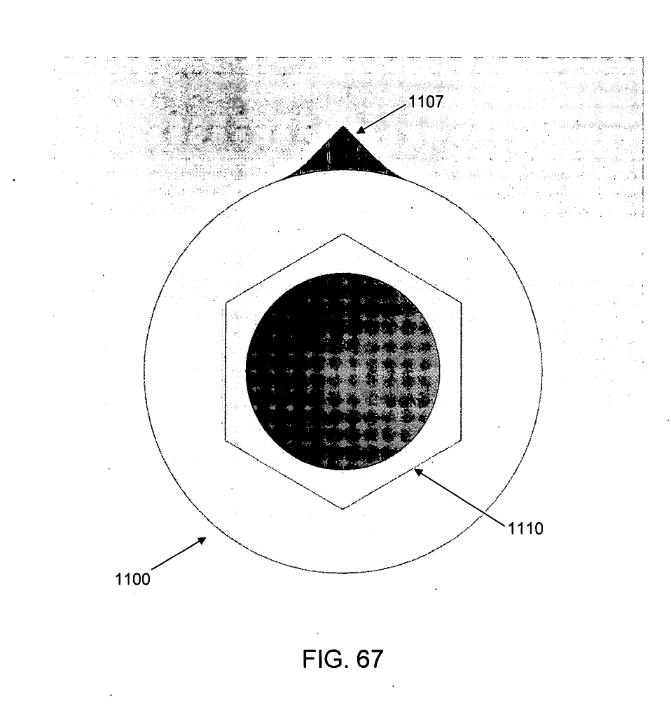

Screw 1100 is shown in detail in FIGS. 55, 58 and 65-67. Screw 1100 generally comprises a body 1102 having threads 1105 and at least one lateral projection 1107 formed thereon, and a driver recess 1110 formed on its proximal end and extending longitudinally into screw 1100.

On account of the foregoing construction, sleeve 1000 and screw 1100 can be used to secure plate 900 to bone as will hereinafter be discussed in further detail.

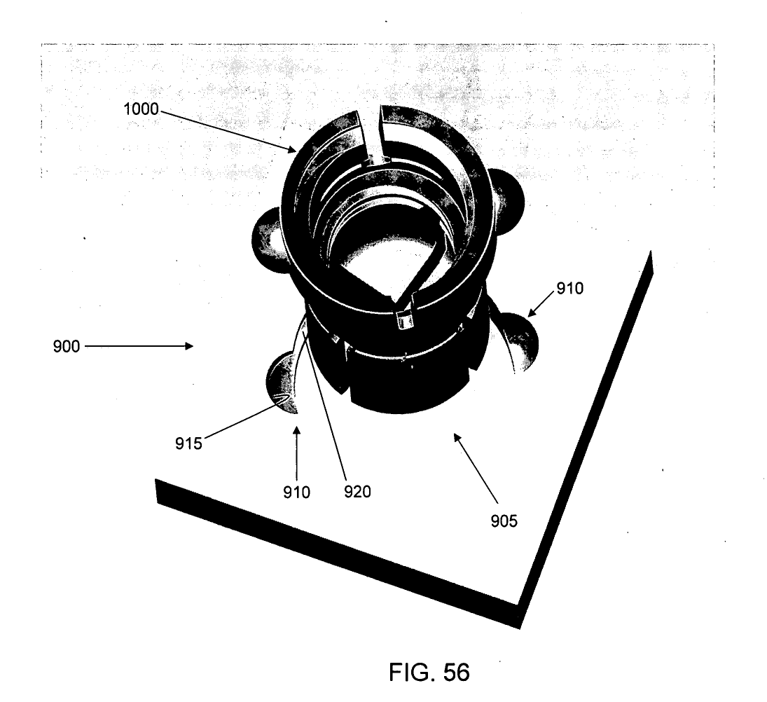

Looking now at FIGS. 56-58, plate 900 is positioned against a bone (not shown) and then sleeve 1000 is passed through plate 900. Sleeve 1000 is passed through plate 900 by advancing sleeve 1000 through bore 905 of plate 900, aligning pivots 1030 of sleeve 1000 with openings 915 of plate 900, and then turning pivots 1030 into undercut 920 of plate 900, whereby to pivotally mount sleeve 1000 to plate 900. Then, screw 1100 is advanced into bore 1020 of sleeve 1000. The advancing screw 1100 causes sleeve 1000 to radially expand so that sleeve 1000 is simultaneously secured to both the bone and to plate 900, thereby securing plate 900 to the bone.

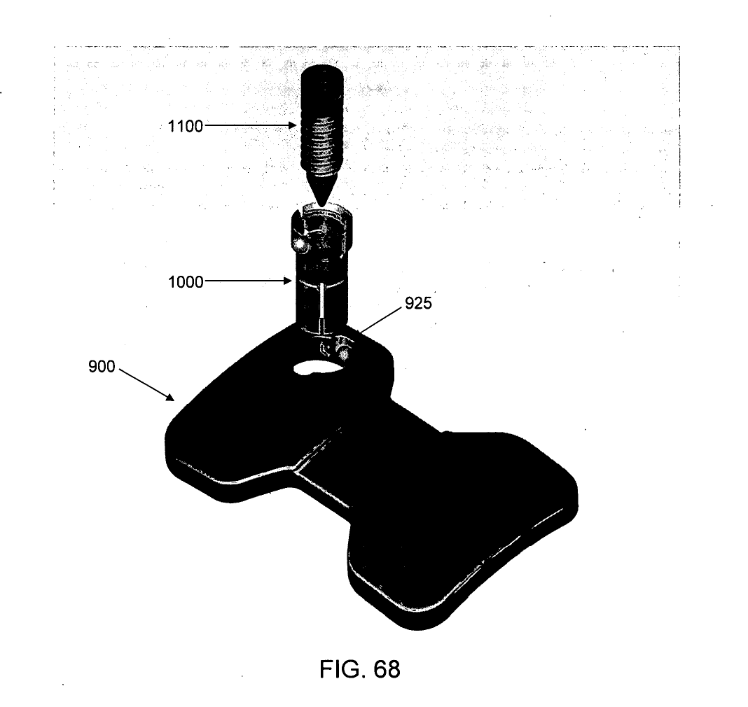

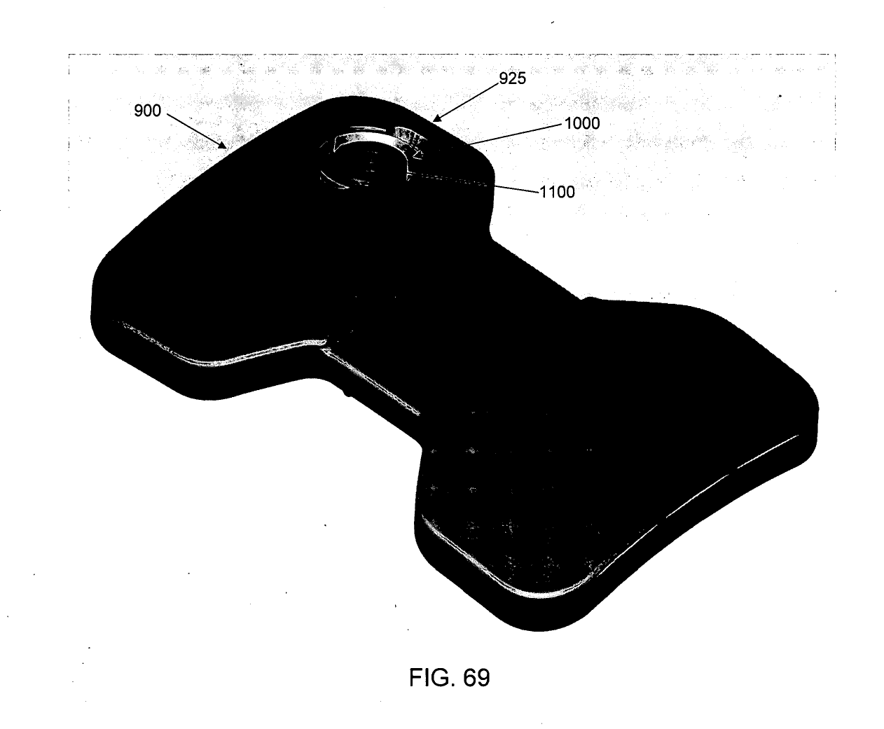

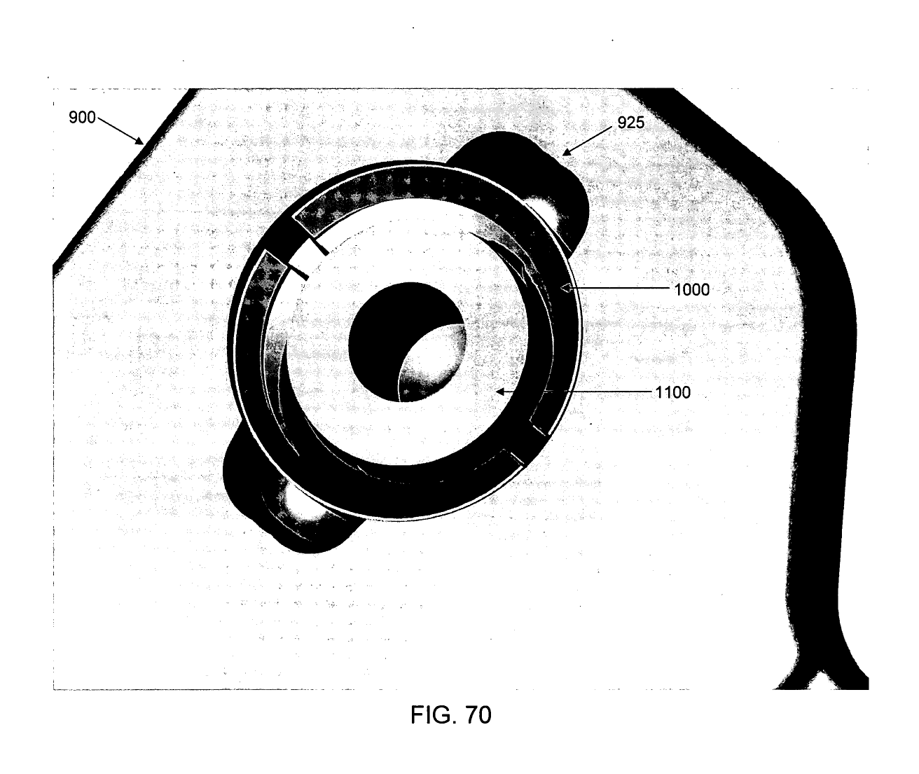

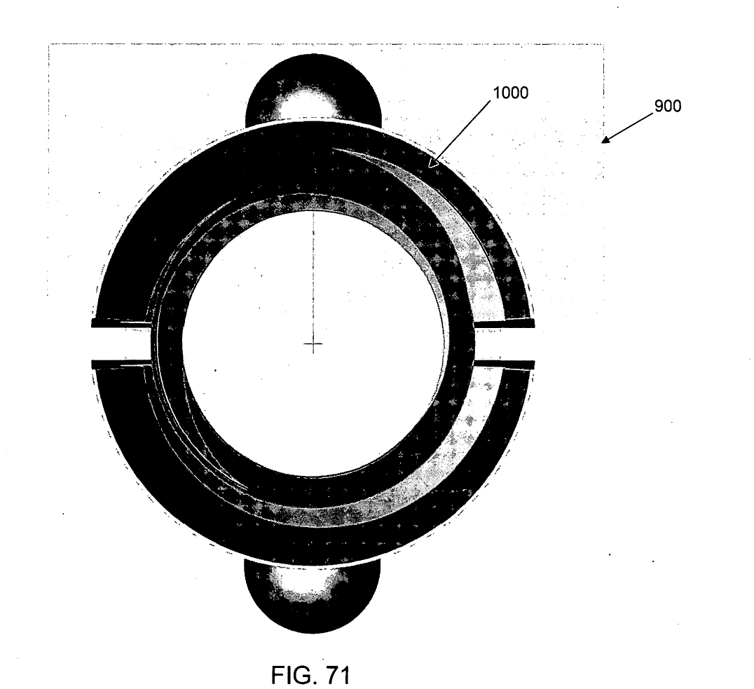

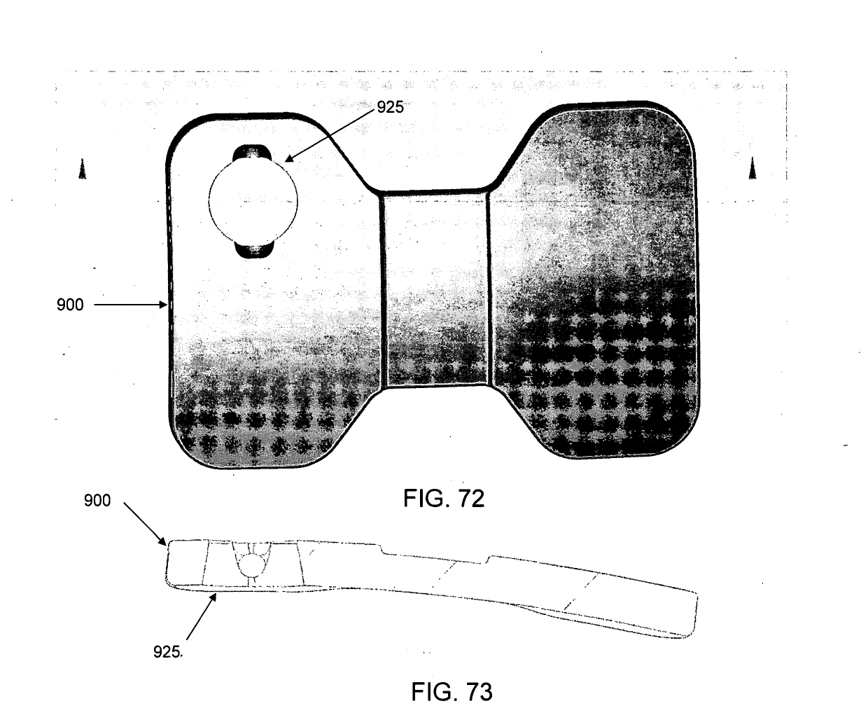

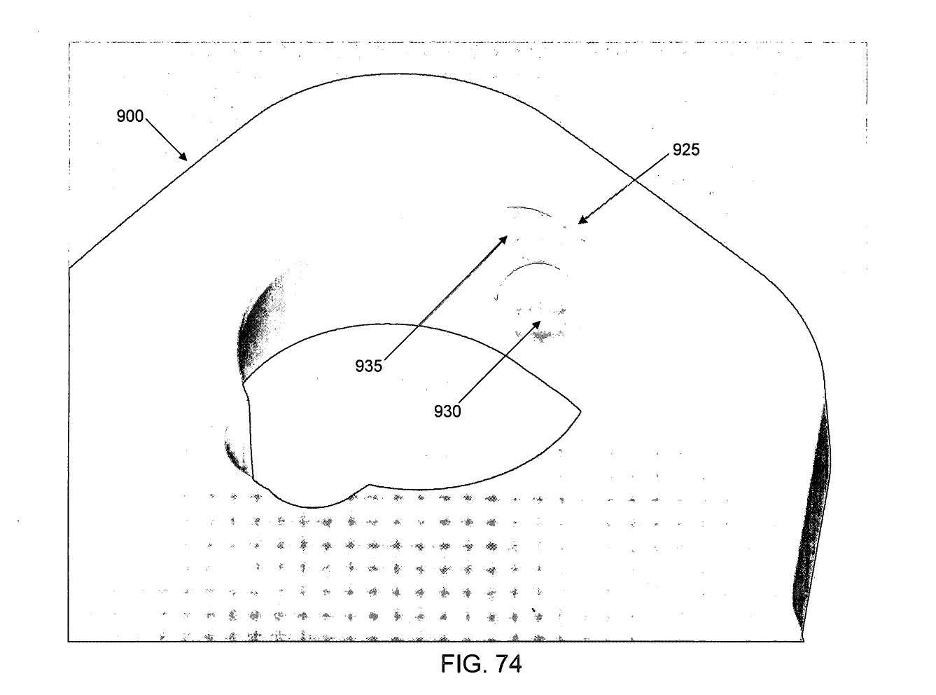

Looking next at FIGS. 68-74, there is shown an alternative construction in which bayonet mount 910 is replaced with a “drop mount” 925. Drop mount 925 generally comprises a pair of seats 930 for receiving pivots 1030 of sleeve 1000 and tapered lead-ins 935 (FIG. 74) leading to each of the seats 930.

In this form of the invention, sleeve 1000 is pivotally fashioned to plate 900 by aligning pivots 1030 with tapered lead-ins 935, and then longitudinally advancing sleeve 1000 so that pivots 1030 movably seat in seats 930, whereby to pivotally mount sleeve 1000 to plate 900 until screw 1100 is advanced into sleeve 1000.

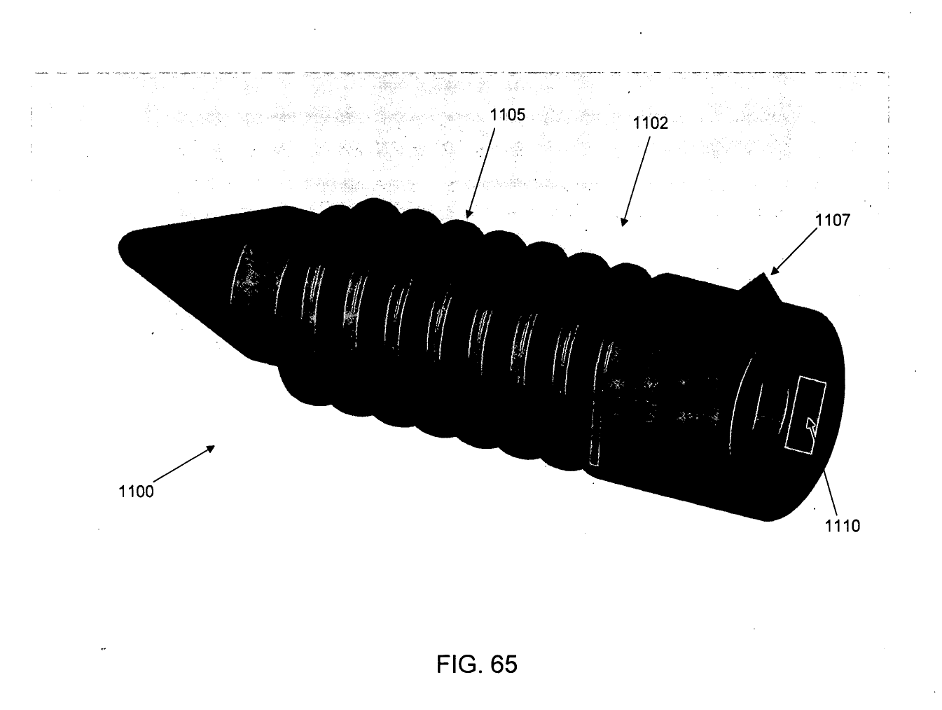

It should also be noted that in these constructions, threads 1105 on screw 1100 (FIG. 65) are “timed” with threads 1023 within sleeve 1000 (FIG. 62) so that screw projection 1107 always lines up approximately 90 degrees from slits 1025 (FIG. 62) of sleeve 1000 when screw 1100 is fully engaged into sleeve 1000, thus expanding sleeve head 1015. Adjustment of the location, size and shape of projection 1107, combined with the size and shape of slits 1025, slots 1035 and enlarged opening 1040, and combined with the size and shape of pivots 1030 and mounts 910 (FIG. 56), 925 (FIG. 74), determine the forces needed to deploy and expand the head of sleeve 1000 into the seats of plate 900. In addition, those features determine the locking force that allows pivotal movement between sleeve 1000 relative to the plate 900.

### Materials

The various components can be formed out of any material or materials consistent with the present invention. Thus, for example, some or all of the components may be formed out of implantable metals (e.g., surgical grade stainless steel, titanium, Nitinol, etc.), implantable plastics, implantable absorbables, etc.

### MODIFICATIONS

It will be understood that many changes in the details, materials, steps and arrangements of parts, which have been herein described and illustrated in order to explain the nature of the invention, may be made by those skilled in the art without departing from the principles and scope of the present invention.

What is claimed is:

1. A surgical system comprising:
an object to be secured to bone, the object comprising an opening extending therethrough; and
a sleeve/screw construction for securing the object to bone, the sleeve/screw construction comprising:
a sleeve adapted for positioning through the opening in the object and into the bone, the sleeve comprising:
a shank having a distal end and a proximal end and an opening extending from the distal end to the proximal end, wherein the opening narrows toward the distal end of the shank, and further wherein the distal end of the shank is slit so as to form a plurality of radially-expandable segments; and
an enlarged head formed at the proximal end of the shank, the enlarged head being formed so as to be radially-expandable;
the sleeve being sized so that when the sleeve is positioned through the opening in the object and into the bone, at least a portion of the radially-expandable segments extend into the bone beyond the cortical bone/cancellous bone interface; and
a screw adapted for positioning through the opening in the sleeve, the screw being sized so as to (i) radially expand the distal end of the sleeve so that the sleeve is secured to the bone, and (ii) radially expand the enlarged head of the sleeve so that the sleeve is secured to the object, whereby to secure the object to the bone.

2. A system according to claim 1 wherein the object comprises a plate.

3. A system according to claim 2 wherein the plate comprises a fracture fixation plate, and further wherein the plate comprises a plurality of openings extending through the plate, the openings being spaced from one another so that at least one opening is disposed on either side of a fracture line.

4. A system according to claim 1 wherein:
the opening in the object comprises (i) a bore passing through the object; (ii) a recess extending into the object, wherein the recess extends parallel to the bore and communicates therewith, and wherein the recess extends radially outward from the bore; and (iii) an undercut communicating with the recess and the bore, wherein the undercut extends radially outward from the bore and circumferentially from the recess; and
the enlarged head of the sleeve is sized to be slidably received with the bore in the object, and further wherein the enlarged head of the sleeve comprises a laterally-extending pivot sized to be slidably received in the recess and the undercut;
whereby the opening of the object and the enlarged head together form a bayonet mount for mounting the sleeve to the object.

5. A system according to claim 4 wherein:
the opening in the object further comprises (i) a second recess extending into the object, wherein the second recess extends parallel to the bore and communicates therewith, and wherein the second recess extends radially outward from the bore; and (ii) a second undercut communicating with the second recess and the bore, wherein the second undercut extends radially outward from the bore and circumferentially from the second recess; and
the enlarged head of the sleeve comprises a second laterally-extending pivot sized to be slidably received in the second recess and the second undercut.

6. A system according to claim 5 wherein the laterally-extending pivot and the second laterally-extending pivot are diametrically-opposed from one another.

7. A system according to claim 1 wherein:
the opening in the object comprises a bore passing through the object; and
a recess extending into the object, wherein the recess extends parallel to the bore and communicates therewith, and wherein the recess extends radially outward from the bore; and
the enlarged head of the sleeve is sized to be slidably received with the bore in the object, and further wherein the enlarged head of the sleeve comprises a laterally-extending pivot sized to be slidably received in the recess;
whereby the opening of the object and the enlarged head together form a drop mount for mounting the sleeve to the object.

8. A system according to claim 7 wherein:
the opening in the object further comprises a second recess extending into the object, wherein the second recess extends parallel to the bore and communicates therewith, and wherein the second recess extends radially outward from the bore; and
the enlarged head of the sleeve comprises a second laterally-extending pivot sized to be slidably received in the second recess.

9. A system according to claim 8 wherein the laterally-extending pivot and the second laterally-extending pivot are diametrically-opposed from one another.

10. A system according to claim 1 wherein the slit at the distal end of the shank of the sleeve terminates in a round hole.

11. A system according to claim 1 wherein the distal end of the shank of the sleeve comprises a plurality of slits, and further wherein each of the slits terminate in round holes.

12. A system according to claim 11 wherein the distal end of the shaft comprises a circumferentially-extending groove communicating with the round holes.

13. A system according to claim 1 wherein the enlarged head of the sleeve comprises a radially-extending slit, whereby to facilitate radial expansion of the enlarged head of the sleeve when the screw is disposed in the sleeve.

14. A system according to claim 13 wherein the radially-extending slit terminates in a round hole.

15. A system according to claim 14 wherein the enlarged head of the sleeve further comprises a slot extending laterally outward from the round hole.

16. A system according to claim 15 wherein the slot is centered on the round hole.

17. A system according to claim 1 wherein the enlarged head of the sleeve comprises a plurality of radially-extending slits, and further wherein each of the radially-extending slits terminate in round holes.

18. A system according to claim 1 wherein the screw is replaced by a ribbed pin.

19. A method for securing an object to bone, the method comprising the steps of:
providing an object having an opening extending therethrough, and providing a sleeve/screw construction for securing the object to bone, the sleeve/screw construction comprising:
a sleeve adapted for positioning through the opening in the object and into the bone, the sleeve comprising:
a shank having a distal end and a proximal end and an opening extending from the distal end to the proximal end, wherein the opening narrows toward the distal end of the shank, and further wherein the distal end of the shank is slit so as to form a plurality of radially-expandable segments;
an enlarged head formed at the proximal end of the shank, the enlarged head being formed so as to be radially-expandable;
the sleeve being sized so that when the sleeve is positioned through the opening in the object and into the bone, at least a portion of the radially-expandable segments extend into the bone beyond the cortical bone/cancellous bone interface; and
a screw adapted for positioning through the opening in the sleeve, the screw being sized so as to (i) radially expand the distal end of the sleeve so that the sleeve is secured to the bone, and (ii) radially expand the enlarged head of the sleeve so that the sleeve is secured to the object, whereby to secure the object to the bone;
positioning the object against the bone;
placing the sleeve through the opening in the object and into the bone; and
positioning the screw in the sleeve so as to secure the sleeve to the bone and to the object, whereby to secure the object to the bone.

20. A method according to claim 19 wherein the object comprises a plate.

21. A method according to claim 20 wherein the plate comprises a fracture fixation plate, and further wherein the plate comprises a plurality of openings extending through the plate, the openings being spaced from one another so that at least one opening is disposed on either side of a fracture line.

22. A method according to claim 19 wherein:
the opening in the object comprises (i) a bore passing through the object; (ii) a recess extending into the object, wherein the recess extends parallel to the bore and communicates therewith, and wherein the recess extends radially outward from the bore; and (iii) an undercut communicating with the recess and the bore, wherein the undercut extends radially outward from the bore and circumferentially from the recess; and
the enlarged head of the sleeve is sized to be slidably received with the bore in the object, and further wherein the enlarged head of the sleeve comprises a laterally-extending pivot sized to be slidably received in the recess and the undercut;
whereby the opening of the object and the enlarged head together form a bayonet mount for mounting the sleeve to the object.

23. A method according to claim 22 wherein:
the opening in the object further comprises (i) a second recess extending into the object, wherein the second recess extends parallel to the bore and communicates therewith, and wherein the second recess extends radially outward from the bore; and (ii) a second undercut communicating with the second recess and the bore, wherein the second undercut extends radially outward from the bore and circumferentially from the second recess; and
the enlarged head of the sleeve comprises a second laterally-extending pivot sized to be slidably received in the second recess and the second undercut.

24. A method according to claim 23 wherein the laterally-extending pivot and the second laterally-extending pivot are diametrically-opposed from one another.

25. A method according to claim 19 wherein:
the opening in the object comprises a bore passing through the object; and a recess extending into the object, wherein the recess extends parallel to the bore and communicates therewith, and wherein the recess extends radially outward from the bore; and
the enlarged head of the sleeve is sized to be slidably received with the bore in the object, and further wherein the enlarged head of the sleeve comprises a laterally-extending pivot sized to be slidably received in the recess;
whereby the opening of the object and the enlarged head together form a drop mount for mounting the sleeve to the object.

26. A method according to claim 25 wherein:
the opening in the object further comprises a second recess extending into the object, wherein the second recess extends parallel to the bore and communicates therewith, and wherein the second recess extends radially outward from the bore; and
the enlarged head of the sleeve comprises a second laterally-extending pivot sized to be slidably received in the second recess.

27. A method according to claim 26 wherein the laterally-extending pivot and the second laterally-extending pivot are diametrically-opposed from one another.

28. A method according to claim 19 wherein the slit at the distal end of the shank of the sleeve terminates in a round hole.

29. A method according to claim 19 wherein the distal end of the shank of the sleeve comprises a plurality of slits, and further wherein each of the slits terminate in round holes.

30. A method according to claim 22 wherein the distal end of the shaft comprises a circumferentially-extending groove communicating with the round holes.

31. A method according to claim 19 wherein the enlarged head of the sleeve comprises a radially-extending slit, whereby to facilitate radial expansion of the enlarged head of the sleeve when the screw is disposed in the sleeve.

32. A method according to claim 32 wherein the radially-extending slit terminates in a round hole.

33. A method according to claim 32 wherein the enlarged head of the sleeve further comprises a slot extending laterally outward from the round hole.

34. A method according to claim 33 wherein the slot is centered on the round hole.

35. A method according to claim 19 wherein the enlarged head of the sleeve comprises a plurality of radially-extending slits, and further wherein each of the radially-extending slits terminate in round holes.

36. A method according to claim 19 wherein the screw is replaced by a ribbed pin.
